(19) United States
(12) Patent Application Publication
(10) Pub. No.: US 2003/0040471 A1
(43) Pub. Date: Feb. 27, 2003

(54) Compositions isolated from skin cells and methods for their use

(76) Inventors: James D. Watson, St. Heliers (NZ); Lorna Strachan, Auckland (NZ); Matthew Sleeman, Weston Colville (GB); Rene Onrust, Auckland (NZ); James G. Murison, Auckland (NZ); Krishanand D. Kumble, Los Altos, CA (US)

Correspondence Address: SPECHMAN LAW GROUP, 1501 WESTERN AVE, SUITE 100, SEATTLE, WA, 98101, US

(21) Appl. No.: 09/866,050

(22) Filed: May 24, 2001

Related U.S. Application Data
(63) Continuation-in-part of application No. 09/312,283, filed on May 14, 1999.
Application No. 09/312,283 is a continuation-in-part of application No. 09/188,930, filed on Nov. 9, 1998, now Pat. No. 6,150,502.
Application No. 09/188,930 is a continuation-in-part of application No. 09/069,726, filed on Apr. 29, 1998, now abandoned.
Application No. 09/188,930 is a continuation-in-part of application No. PCT/NZ99/00051, filed on Apr. 29, 1999.
(60) Provisional application No. 60/206,650, filed on May 24, 2000.
(60) Provisional application No. 60/221,232, filed on Jul. 25, 2000.

(51) Int. Cl. A61K 38/17; C12P 21/02; C12N 5/06; C07H 21/04; C07K 14/435

(52) U.S. Cl. 514/12; 530/350; 536/23.5; 435/320.1; 435/325; 435/69.1

(57) ABSTRACT

Isolated polynucleotides encoding polypeptides expressed in mammalian skin cells are provided, together with expression vectors and host cells comprising such isolated polynucleotides. Methods for the use of such polynucleotides and polypeptides are also provided.

### REFERENCE TO RELATED APPLICATIONS

This application is a continuation-in-part of U.S. application Ser. No. 09/312,283, filed May 14, 1999, which is a continuation-in-part of U.S. application Ser. No. 09/188,930, filed Nov. 9, 1998, which is a continuation-in-part of U.S. application Ser. No. 09/069,726, filed Apr. 29, 1998, and claims priority to International Patent Application No. PCT/NZ99/00051, filed Apr. 29, 1999, U.S. Provisional Application No. 60/206,650, filed May 24, 2000, and U.S. Provisional Application No. 60/221,232, filed Jul. 25, 2000.

### TECHNICAL FIELD OF THE INVENTION

This invention relates to polynucleotides, polypeptides, polypeptides expressed in skin cells, and various methods for treating a patient involving administration of a polypeptide or polynucleotide of the present invention.

### BACKGROUND OF THE INVENTION

The skin is the largest organ in the body and serves as a protective cover. The loss of skin, as occurs in a badly burned person, may lead to death owing to the absence of a barrier against infection by external microbial organisms, as well as loss of body temperature and body fluids.

Skin tissue is composed of several layers. The outermost layer is the epidermis which is supported by a basement membrane and overlies the dermis. Beneath the dermis is loose connective tissue and fascia which cover muscles or bony tissue. The skin is a self-renewing tissue in that cells are constantly being formed and shed. The deepest cells of the epidermis are the basal cells, which are enriched in cells capable of replication. Such replicating cells are called progenitor or stem cells. Replicating cells in turn give rise to daughter cells called ‘transit amplifying cells’. These cells undergo differentiation and maturation into keratinocytes (mature skin cells) as they move from the basal layer to the more superficial layers of the epidermis. In the process, keratinocytes become comified and are ultimately shed from the skin surface. Other cells in the epidermis include melanocytes which synthesize melanin, the pigment responsible for protection against sunlight. The Langerhans cell also resides in the epidermis and functions as a cell which processes foreign proteins for presentation to the immune system.

The dermis contains nerves, blood and lymphatic vessels, fibrous and fatty tissue. Within the dermis are fibroblasts, macrophages and mast cells. Both the epidermis and dermis are penetrated by sweat, or sebaceous glands and hair follicles. Each strand of hair is derived from a hair follicle. When hair is plucked out, the hair re-grows from epithelial cells directed by the dermal papillae of the hair follicle.

When the skin surface is breached, for example in a wound, the stem cells proliferate and daughter keratinocytes migrate across the wound to reseal the tissues. The skin cells therefore possess genes activated in response to trauma. The products of these genes include several growth factors, such as epidermal growth factor, which mediate the proliferation of skin cells. The genes that are activated in the skin, and the protein products of such genes, may be developed as agents for the treatment of skin wounds. Additional growth factors derived from skin cells may also influence growth of other cell types. As skin cancers are a disorder of the growth of skin cells, proteins derived from skin that regulate cellular growth may be developed as agents for the treatment of skin cancers. Skin derived proteins that regulate the production of melanin may be useful as agents, which protect skin against unwanted effects of sunlight.

Keratinocytes are known to secrete cytokines and express various cell surface proteins. Cytokines and cell surface molecules are proteins, which play an important role in the inflammatory response against infection, and also in autoimmune diseases affecting the skin. Genes and their protein products that are expressed by skin cells may thus be developed into agents for the treatment of inflammatory disorders affecting the skin.

Hair is an important part of a person's individuality. Disorders of the skin may lead to hair loss. Alopecia areata is a disease characterized by the patchy loss of hair over the scalp. Total baldness is a side effect of drug treatment for cancer. The growth and development of hair is mediated by the effects of genes expressed in skin and dermal papillae. Such genes and their protein products may be usefully developed into agents for the treatment of disorders of the hair follicle.

New treatments are required to hasten the healing of skin wounds, to prevent the loss of hair, enhance the re-growth of hair or removal of hair, and to treat autoimmune and inflammatory skin diseases more effectively and without adverse effects. More effective treatments of skin cancers are also required. There thus remains a need in the art for the identification and isolation of genes encoding proteins expressed in the skin, for use in the development of therapeutic agents for the treatment of disorders including those associated with skin.

### SUMMARY OF THE INVENTION

The present invention provides polypeptides and functional portions of polypeptides, which may be expressed in skin cells, together with polynucleotides encoding such polypeptides or functional portions thereof, expression vectors and host cells comprising such polynucleotides, and methods for their use.

In specific embodiments, isolated polynucleotides are provided that comprise a polynucleotide selected from the group consisting of (a) sequences recited in SEQ ID NOS: 1-119, 198-276, 349-372, 399-405, 410-412, 416, 418-455, 464, 466-487, 510, 511 and 514-623; (b) complements of the sequences recited in SEQ ID NOS: 1-119, 198-276, 349-372, 399-405, 410-412, 416, 418-455, 464, 466-487, 510, 511 and 514-623; (c) reverse complements of the sequences recited in SEQ ID NOS: 1-119, 198-276, 349-372, 399-405, 410-412, 416, 418-455, 464, 466-487, 510, 511 and 514-623; (d) reverse sequences of the sequences recited in SEQ ID NOS: 1-119, 198-276, 349-372, 399-405, 410-412, 416, 418-455, 464, 466-487, 510, 511 and 514-623; (e) sequences having a 99% probability of being the same as a sequence of (a)-(d); and (f) sequences having at least 50%, 75%, 90% or 95% identity to a sequence of (a)-(d).

In further embodiments, the present invention provides isolated polypeptides comprising an amino acid sequence selected from the group consisting of: (a) sequences provided in SEQ ID NOS: 120-197, 275-348, 373-398, 406-409, 413-415, 417, 456-463, 465, 488-509, 512, 513 and 624-725, and (b) sequences having at least 50%, 75%, 90% or 95% identity to a sequence provided in SEQ ID NOS: 120-197, 275-348, 373-398, 406-409, 413-415, 417, 456-463, 465, 488-509, 512, 513 and 624-725, together with isolated polynucleotides encoding such polypeptides. Isolated polypeptides which comprise at least a functional portion of a polypeptide comprising an amino acid sequence selected from the group consisting of: (a) sequences provided in SEQ ID NOS: 120-197, 275-348, 373-398, 406-409, 413-415, 417, 456-463, 465, 488-509, 512, 513 and 624-725; and (b) sequences having 50%, 75%, 90% or 95% identity to a sequence of SEQ ID NOS: 120-197, 275-348, 373-398, 406-409, 413-415, 417, 456-463, 465, 488-509, 512, 513 and 624-725, are also provided.

In related embodiments, the present invention provides expression vectors comprising the above polynucleotides, together with host cells transformed with such vectors.

In a further aspect, the present invention provides a method of stimulating keratinocyte growth and motility, inhibiting the growth of epithelial-derived cancer cells, inhibiting angiogenesis and vascularization of tumors, or modulating the growth of blood vessels in a subject, comprising administering to the subject a composition comprising an isolated polypeptide, wherein the polypeptide comprises an amino acid sequence selected from the group consisting of (a) sequences provided in SEQ ID NOS: 187, 196, 342, 343, 395, 397 and 398; and (b) sequences having at least 50%, 75%, 90% or 95% identity to a sequence provided in SEQ ID NOS: 187, 196, 342, 343, 395, 397 and 398.

Methods for modulating skin inflammation in a subject are also provided, the methods comprising administering to the subject a composition comprising an isolated polypeptide, wherein the polypeptide comprises an amino acid sequence selected from the group consisting of: (a) sequences provided in SEQ ID NOS: 338 and 347; and (b) sequences having at least 50%, 75%, 90% or 95% identity to a sequence provided in SEQ ID NOS: 338 and 347. In an additional aspect, the present invention provides methods for stimulating the growth of epithelial cells in a subject. Such methods comprise administering to the subject a composition comprising an isolated polypeptide including an amino acid sequence selected from the group consisting of (a) sequences provided in SEQ ID NOS: 129 and 348; and (b) sequences having at least 50%, 75%, 90% or 95% identity to a sequence provided in SEQ ID NOS: 129 and 348.

In yet a further aspect, methods for inhibiting the binding of HIV-1 to leukocytes, for the treatment of an inflammatory disease or for the treatment of cancer in a subject are provided, the methods comprising administering to the subject a composition comprising an isolated polypeptide including an amino acid sequence selected from the group consisting of (a) sequences provided in SEQ ID NOS: 340, 344, 345 and 346; and (b) sequences having at least 50%, 75%, 90% or 95% identity to a sequence provided in SEQ ID NOS: 340, 344, 345 and 346.

As detailed below, the isolated polynucleotides and polypeptides of the present invention may be usefully employed in the preparation of therapeutic agents for the treatment of skin disorders.

The above-mentioned and additional features of the present invention, together with the manner of obtaining them, will be best understood by reference to the following more detailed description. All references disclosed herein are incorporated herein by reference in their entirety as if each was incorporated individually.

### BRIEF DESCRIPTION OF THE DRAWINGS

FIG. 1 shows the results of a Northern analysis of the distribution of huTR1 mRNA in human tissues. Key: He, Heart; Br, Brain; PI, Placenta; Lu, Lung; Li, Liver; SM, Skeletal muscle; Ki, Kidney; Sp, Spleen; Th, Thymus; Pr, Prostate; Ov, Ovary.

FIG. 2 shows the results of a MAP kinase assay of muTR1a and huTR1a. MuTR1a (500 ng/ml), huTR1a (10 ng/ml) or LPS (3pg/ml) were added as described in the text.

FIG. 3 shows the stimulation of growth of neonatal foreskin keratinocytes by muTR1 a.

FIG. 4 shows the stimulation of growth of the transformed human keratinocyte cell line HaCaT by muTR1a and huTR1a.

FIG. 5 shows the inhibition of growth of the human epidermal carcinoma cell line A431 by muTR1a and huTR1a.

FIG. 6 shows the inhibition of IL-2 induced growth of concanavalin A-stimulated murine splenocytes by KS2a.

FIG. 7 shows the stimulation of growth of rat intestinal epithelial cells (IEC-18) by a combination of KS3a plus apo-transferrin.

FIG. 8 illustrates the oxidative burst effect of TR-1 (100 ng/ml), muKS1 (100 ng/ml), SDF1α(100 ng/ml), and fMLP (10 μM) on human PBMC.

FIG. 9 shows the chemotactic effect of muKS1 and SDF-1α on THP-1 cells.

FIG. 10 shows the induction of cellular infiltrate in C3H/HeJ mice after intraperitoneal injections with muKS1 (50 μg), GV14B (50 μg) and PBS.

FIG. 11 demonstrates the induction of phosphorylation of ERK1 and ERK2 in CV1/EBNA and HeLa cell lines by huTR1a.

FIG. 12 shows the huTR1 mRNA expression in HeLa cells after stimulation by muTR1, huTR1, huTGFα and PBS (100 ng/ml each).

FIG. 13 shows activation of the SRE by muTR1a in PC-12 (FIG. 13A) and HaCaT (FIG. 13B) cells.

FIG. 14 shows the inhibition of huTR1a mediated growth on HaCaT cells by an antibody to the EGF receptor.

FIG. 15A shows the nucleotide sequence of KS1 cDNA (SEQ ID NO: 464) along with the deduced amino acid sequence (SEQ ID NO: 465) using single letter code. The 5′ UTR is indicated by negative numbers. The underlined NH₂-terminal amino acids represent the predicted leader sequence and the stop codon is denoted by ***. The poly-adenylation signal is marked by a double underline. FIG. 15B shows a comparison of the complete open reading frame of KS1 (referred to in FIG. 15B as KLF-1) with its human homologue BRAK and with the mouse a-chemokines mCrg-2, mMig, mSDF-1, mBLC, mMIP2, mKC and mLIX. An additional five residues are present in KS1 and BRAK between cysteine 3 and cysteine 4 that have not previously been described for chemokines.

### DETAILED DESCRIPTION OF THE INVENTION

In one aspect, the present invention provides polynucleotides that were isolated from mammalian skin cells. As used herein, the term “polynucleotide” means a single or double-stranded polymer of deoxyribonucleotide or ribonucleotide bases and includes DNA and RNA molecules, both sense and anti-sense strands. The term comprehends cDNA, genomic DNA, recombinant DNA and wholly or partially synthesized nucleic acid molecules. A polynucleotide may consist of an entire gene, or a portion thereof A gene is a DNA sequence that codes for a functional protein or RNA molecule. Operable anti-sense polynucleotides may comprise a fragment of the corresponding polynucleotide, and the definition of “polynucleotide” therefore includes all operable anti-sense fragments. Anti-sense polynucleotides and techniques involving anti-sense polynucleotides are well known in the art and are described, for example, in Robinson-Benion et al., “Anti-sense Techniques,” Methods in Enzymol. 254(23):363-375, 1995; and Kawasaki et al., Artific. Organs 20(8):836-848, 1996.

Identification of genomic DNA and heterologous species DNAs can be accomplished by standard DNA/DNA hybridization techniques, under appropriately stringent conditions, using all or part of a cDNA sequence as a probe to screen an appropriate library. Alternatively, PCR techniques using oligonucleotide primers that are designed based on known genomic DNA, cDNA and protein sequences can be used to amplify and identify genomic and cDNA sequences. Synthetic DNAs corresponding to the identified sequences and variants may be produced by conventional synthesis methods. All the polynucleotides provided by the present invention are isolated and purified, as those terms are commonly used in the art.

In specific embodiments, the polynucleotides of the present invention comprise a sequence selected from the group consisting of sequences provided in SEQ ID NOS: 1-119, 198-274, 349-372, 399-405, 410-412, 416, 418-455, 464, 466-487, 510, 511 and 514-623, and variants of the sequences of SEQ ID NOS: 1-119, 198-274, 349-372, 399-405, 410-412, 416, 418-455, 464, 466-487, 510, 511 and 514-623. Polynucleotides that comprise complements of such sequences, reverse complements of such sequences, or reverse sequences of such sequences, together with variants of such sequences, are also provided.

The definition of the terms “complement,” “reverse complement,” and “reverse sequence,” as used herein, is best illustrated by the following example. For the sequence 5′ AGGACC 3′, the complement, reverse complement, and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

As used herein, the term “complement” refers to sequences that are fully complementary to a sequence disclosed herein.

In another aspect, the present invention provides isolated polypeptides and functional portions of polypeptides encoded, or partially encoded, by the above polynucleotides. As used herein, the term “polypeptide” encompasses amino acid chains of any length, including full length proteins, wherein the amino acid residues are linked by covalent peptide bonds. The term “polypeptide encoded by a polynucleotide” as used herein, includes polypeptides encoded by a polynucleotide which comprises a partial isolated DNA sequence provided herein. In specific embodiments, the inventive polypeptides comprise an amino acid sequence selected from the group consisting of sequences provided in SEQ ID NOS: 120-197, 275-348, 373-398, 406-409, 413-415, 417, 456-463, 465, 488-509, 512, 513 and 624-725, as well as variants of such sequences.

Polypeptides of the present invention may be produced recombinantly by inserting a DNA sequence that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast, and higher eukaryotic cells. Preferably, the host cells employed are E. coli , insect, yeast, or a mammalian cell line such as COS or CHO. The DNA sequences expressed in this manner may encode naturally occurring polypeptides, portions of naturally occurring polypeptides, or other variants thereof In a related aspect, polypeptides are provided that comprise at least a functional portion of a polypeptide having an amino acid sequence selected from the group consisting of sequences provided in SEQ ID NOS: 120-197, 275-348, 373-398, 406-409, 413-415, 417, 456-463, 465, 488-509, 512-513 and 624-725, and variants thereof As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may also be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed site-specific mutagenesis (Kunkel, T., Proc. Natl. Acad. Sci. USA 82:488-492, 1985). Sections of DNA sequence may also be removed using standard techniques to permit preparation of truncated polypeptides.

In general, the polypeptides disclosed herein are prepared in an isolated, substantially pure, form. Preferably, the polypeptides are at least about 80% pure, more preferably at least about 90% pure, and most preferably at least about 99% pure. In certain preferred embodiments, described in detail below, the isolated polypeptides are incorporated into pharmaceutical compositions or vaccines for use in the treatment of skin disorders.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. In certain preferred embodiments, variants of the inventive sequences retain certain, or all, of the functional characteristics of the inventive sequence. Variant sequences (polynucleotide or polypeptide) preferably exhibit at least 50%, more preferably at least 75%, and most preferably at least 90% or 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotide or polypeptide sequences may be aligned, and percentages of identical nucleotides in a specified region may be determined against another polynucleotide or polypeptide, using computer algorithms that are publicly available. Two exemplary algorithms for aligning and identifying the similarity of polynucleotide sequences are the BLASTN and FASTA algorithms. The alignment and similarity of polypeptide sequences may be examined using the BLASTP and algorithm. BLASTX and FASTX algorithms compare nucleotide query sequences translated in all reading frames against polypeptide sequences. The BLASTN, BLASTP and BLASTX algorithms are available on the NCBI anonymous FTP server (ftp://ncbi.nlm.nih.gov) under/blast/executables/and are available from the National Center for Biotechnology Information (NCBI), National Library of Medicine, Building 38A, Room 8N805, Bethesda, Md. 20894 USA.

The FASTA and FASTX algorithms are available on the Internet at the ftp site ftp://ftp.Virginia.edu/pub/. The FASTA software package is also available from the University of Virginia by contacting David Hudson, Assistant Provost for Research, University of Virginia, PO Box 9025, Charlottesville, Va. 22906-9025. The FASTA algorithm, set to the default parameters described in the documentation and distributed with the algorithm, may be used in the determination of polynucleotide variants. The readme files for FASTA and FASTX v1.0x that are distributed with the algorithms describe the use of the algorithms and describe the default parameters. The use of the FASTA and FASTX algorithms is also described in Pearson, and Lipman, Proc. Natl. Acad. Sci. USA 85:2444-2448, 1988; and Pearson, Methods in Enzymol. 183:63-98, 1990.

The BLASTN algorithm version 2.0.4 [Feb. 24, 1998], 2.0.6 [Sep. 16, 1998] and 2.0.11 [Jan. 20, 2000], set to the default parameters described in the documentation and distributed with the algorithm, is preferred for use in the determination of polynucleotide variants according to the present invention. The BLASTP algorithm version 2.0.4, 2.0.6 and 2.0.11, set to the default parameters described in the documentation and distributed with the algorithm, is preferred for use in the determination of polypeptide variants according to the present invention. The use of the BLAST family of algorithms, including BLASTN, BLASTP and BLASTX is described in the publication of Altschul, et al., Nucleic Acids Res. 25:33 89-3402, 1997.

The following running parameters are preferred for determination of alignments and similarities using BLASTN that contribute to the E values and percentage identity for polynucleotides: Unix running command with default parameters thus: blastall -p blastn -d embldb -e 10-G 0-E 0 -r 1 -v 30 -b 30 -i queryseq -o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; -o BLAST report Output File [File Out] Optional. The following running parameters are preferred for determination of alignments and similarities using BLASTP that contribute to the E values and percentage identity for polypeptides: blastaH -p blastp -d swissprotdb -e 10-G 1-E 11 -r 1 -v 30 -b 30 -i queryseq -o results; and the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23 nucleotide hit includes 21 identical nucleotides, one gap and one different nucleotide. The percentage identity of the queried polynucleotide to the hit in the EMBL database is thus 21/220 times 100, or 9.5%. The identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being the same. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being the same as the polypeptide of the present invention, measured as having an E value of 0.01 or less using the BLASTP algorithm set at the default parameters.

Variant polynucleotide sequences will generally hybridize to the recited polynucleotide sequences under stringent conditions. As used herein, “stringent conditions” refers to prewashing in a solution of 6× SSC, 0.2% SDS; hybridizing at 65° C., 6× SSC, 0.2% SDS overnight; followed by two washes of 30 minutes each in 1× SSC, 0.1% SDS at 65° C. and two washes of 30 minutes each in 0.2× SSC, 0.1% SDS at 65° C.

As used herein, the term “x-mer,” with reference to a specific value of “x,” refers to a polynucleotide or polypeptide, respectively, comprising at least a specified number (“x”) of contiguous residues of any of the polynucleotides provided in SEQ ID NO: 1-119, 198-274, 349-372, 399-405, 410-412, 416, 418-455, 464, 466-487, 510, 511 and 514-623; or any of the polypeptides set out in SEQ ID NO: 120-197, 275-348, 373-398, 406-409, 413-415, 417, 456-463, 465, 488-509, 512, 513 and 624-725. The value of x may be from about 20 to about 600, depending upon the specific sequence.

Polynucleotides of the present invention comprehend polynucleotides comprising at least a specified number of contiguous residues (x-mers) of any of the polynucleotides identified as SEQ ID NO: 1-119, 198-274, 349-372, 399-405, 410-412, 416, 418-455, 464, 466-487, 510, 511 and 514-623, or their variants. Polypeptides of the present invention comprehend polypeptides comprising at least a specified number of contiguous residues (x-mers) of any of the polypeptides identified as SEQ ID NO: 120-197, 275-348, 373-398, 406-409, 413-415, 417, 456-463, 465, 488-509, 512, 513 and 624-725. According to preferred embodiments, the value of x is at least 20, more preferably at least 40, more preferably yet at least 60, and most preferably at least 80. Thus, polynucleotides of the present invention include polynucleotides comprising a 20-mer, a 40-mer, a 60-mer, an 80-mer, a 100-mer, a 120-mer, a 150-mer, a 180-mer, a 220-mer, a 250-mer; or a 300-mer, 400-mer, 500-mer or 600-mer of a polynucleotide provided in SEQ ID NOS: 1-119, 198-274, 349-372, 399-405, 410-412, 416, 418-455, 464, 466-487, 510, 511 and 514-623, or of a variant of one of the polynucleotides provided in SEQ ID NO: 1-119, 198-274, 349-372, 399-405, 410-412, 416, 418-455, 464, 466-487, 510, 511 and 514-623. Polypeptides of the present invention include polypeptides comprising a 20-mer, a 40-mer, a 60-mer, an 80-mer, a 100-mer, a 120-mer, a 150-mer, a 180-mer, a 220-mer, a 250-mer; or a 300-mer, 400-mer, 500-mer or 600-mer of a polypeptide provided in SEQ ID NOS: 120-197, 275-348, 373-398, 406-409, 413-415, 417, 456-463, 465, 488-509, 512, 513 and 624-725, or of a variant of one of the polypeptides provided in SEQ ID NOS: 120-197, 275-348, 373-398, 406-409, 413-415, 417, 456-463, 465, 488-509, 512, 513 and 624-725.

The inventive polynucleotides may be isolated by high throughput sequencing of cDNA libraries prepared from mammalian skin cells as described below in Example 1. Alternatively, oligonucleotide probes based on the sequences provided in SEQ ID NOS: 1-119, 198-274, 349-372, 399-405, 410-412, 416, 418-455, 464, 466-487, 510, 511 and 514-623 can be synthesized and used to identify positive clones in either cDNA or genomic DNA libraries from mammalian skin cells by means of hybridization or polymerase chain reaction (PCR) techniques. Probes can be shorter than the sequences provided herein but should be at least about 10, preferably at least about 15 and most preferably at least about 20 nucleotides in length. Hybridization and PCR techniques suitable for use with such oligonucleotide probes are well known in the art (see, for example, Mullis, et al., Cold Spring Harbor Symp. Quant. Biol., 51:263, 1987; Erlich, ed., PCR Technology, Stockton Press: NY, 1989; (Sambrook, J, Fritsch, E F and Maniatis, T, eds., Molecular Cloning: A Laboratory Manual, 2nd ed., Cold Spring Harbor Laboratory Press, Cold Spring Harbor: New York, 1989). Positive clones may be analyzed by restriction enzyme digestion, DNA sequencing or the like.

In addition, DNA sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

Since the polynucleotide sequences of the present invention have been derived from skin, they likely encode proteins that have important roles in growth and development of skin, and in responses of skin to tissue injury and inflammation as well as disease states. Some of the polynucleotides contain sequences that code for signal sequences, or transmembrane domains, which identify the protein products as secreted molecules or receptors. Such protein products are likely to be growth factors, cytokines, or their cognate receptors. Several of the polypeptide sequences have more than 25% similarity to known biologically important proteins and thus are likely to represent proteins having similar biological functions.

In particular, the inventive polypeptides have important roles in processes such as: induction of hair growth; differentiation of skin stem cells into specialized cell types; cell migration; cell proliferation and cell-cell interaction. The polypeptides are important in the maintenance of tissue integrity, and thus are important in processes such as wound healing. Some of the disclosed polypeptides act as modulators of immune responses, especially since immune cells are known to infiltrate skin during tissue insult causing growth and differentiation of skin cells. In addition, many polypeptides are immunologically active, making them important therapeutic targets in a whole range of disease states not only within skin, but also in other tissues of the body. Antibodies to the polypeptides of the present invention and small molecule inhibitors related to the polypeptides of the present invention may also be used for modulating immune responses and for treatment of diseases according to the present invention.

In one aspect, the present invention provides methods for using one or more of the inventive polypeptides or polynucleotides to treat disorders in a patient. As used herein, a “patient” refers to any warm-blooded animal, preferably a human.

In this aspect, the polypeptide or polynucleotide is generally present within a pharmaceutical or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and a non-specific immune response amplifier, such as an adjuvant or a liposome, into which the polypeptide is incorporated.

Alternatively, a pharmaceutical or immunogenic composition of the present invention may contain DNA encoding one or more polypeptides as described above, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus-Calmette-Guerin) that expresses an immunogenic portion of the polypeptide on its cell surface. In a preferred embodiment, the DNA may be introduced using a viral expression system (e.g., vaccinia or other poxyirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Routes and frequency of administration, as well as dosage, vary from individual to individual. In general, the pharmaceutical and immunogenic compositions may be administered by injection (e.g., intradermal, intramuscular, intravenous, or subcutaneous), intranasally (e.g., by aspiration) or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the patient, but will typically range from about 0.1 ml to about 5 ml.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a lipid, a wax, or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of the invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis, or Mycobacterium tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A, and Quil A.

The polynucleotides of the present invention may also be used as markers for tissue, as chromosome markers or tags, in the identification of genetic disorders, and for the design of oligonucleotides for examination of expression patterns using techniques well known in the art, such as the microarray technology available from Affymetrix (Santa Clara, Calif.). Partial polynucleotide sequences disclosed herein may be employed to obtain full length genes by, for example, screening of DNA expression libraries using hybridization probes or PCR primers based on the inventive sequences.

The polypeptides provided by the present invention may additionally be used in assays to determine biological activity, to raise antibodies, to isolate corresponding ligands or receptors, in assays to quantitatively determine levels of protein or cognate corresponding ligand or receptor, as anti-inflammatory agents, and in compositions for skin, connective tissue and/or nerve tissue growth or regeneration. The present invention further provides methods for modulating expression of the inventive polypeptides, for example by inhibiting translation of the relevant polynucleotide. Translation of the relevant polynucleotide may be inhibited, for example, by introducing anti-sense expression vectors; by introducing antisense oligodeoxyribonucleotides or antisense phosphorothioate oligodeoxyribonucleotides; by introducing antisense oligoribonucleotides or antisense phosphorothioate oligoribonucleotides; or by other means which are well known in the art. Cell permeation and activity of antisense oligonucleotides can be enhanced by appropriate chemical modifications, such as the use of phenoxazine-substituted C-5 propynyl uracil oligonucleotides (Flanagan et al., (1999) Nat. Biotechnol. 17 (1): 48-52) or 2′-O-(2-methoxy) ethyl (2′-MOE)-oligonucleotides (Zhang et al., (2000) Nat. Biotechnol. 18: 862-867). The use of techniques involving antisense polynucleotides is well known in the art and is described, for example, in Robinson-Benion et al. (1995), Antisense techniques, Methods in Enzymol. 254 (23): 363-375 and Kawasaki et al. (1996), Artific. Organs 20 (8): 836-848.

The following Examples are offered by way of illustration and not by way of limitation.

### EXAMPLE 1

### Isolation of cDNA Sequences from Skin Cell Expression Libraries

The cDNA sequences of the present invention were obtained by high-throughput sequencing of cDNA expression libraries constructed from specialized rodent or human skin cells as shown in Table 1.

TABLE 1

| Library | Skin cell type | Source |
|---|---|---|
| DEPA | dermal papilla | rat |
| SKTC | keratinocytes | human |
| HNFF | neonatal foreskin fibroblast | human |
| MEMS | embryonic skin | mouse |
| KSCL | keratinocyte stem cell | mouse |
| TRAM | transit amplifying cells | mouse |
| MFSE | epidermis | mouse |
| HLEA | small epithelial airway cells | human |
| HLEB | small epithelial airway cells | human |
| HNKA | NK cells | human |

These cDNA libraries were prepared as described below.

cDNA Library from Dermal Papilla (DEPA)

Dermal papilla cells from rat hair vibrissae (whiskers) were grown in culture and the total RNA extracted from these cells using established protocols. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library was then prepared from the mRNA by reverse transcriptase synthesis using a Lambda ZAP cDNA library synthesis kit (Stratagene).

cDNA Library from Keratinocytes (SKTC)

Keratinocytes obtained from human neonatal foreskins (Mitra, R and Nikoloff, B in Handbook of Keratinocyte Methods, pp. 17-24, 1994) were grown in serum-free KSFM (BRL Life Technologies) and harvested along with differentiated cells (10⁸ cells). Keratinocytes were allowed to differentiate by addition of fetal calf serum at a final concentration of 10% to the culture medium and cells were harvested after 48 hours. Total RNA was isolated from the two cell populations using TRIzol Reagent (BRL Life Technologies) and used to obtain mRNA using a Poly(A) Quik mRNA isolation kit (Stratagene). cDNAs expressed in differentiated keratinocytes were enriched by using a PCR-Select cDNA Subtraction Kit (Clontech, Palo Alto, Calif.). Briefly, mRNA was obtained from either undifferentiated keratinocytes (“driver mRNA”) or differentiated keratinocytes (“tester mRNA”) and used to synthesize cDNA. The two populations of cDNA were separately digested with RsaI to obtain shorter, blunt-ended molecules. Two tester populations were created by ligating different adaptors at the cDNA ends and two successive rounds of hybridization were performed with an excess of driver cDNA. The adaptors allowed for PCR amplification of only the differentially expressed sequences which were then ligated into T-tailed pBluescript (Hadjeb, N and Berkowitz, Ga., BioTechniques 20:20-22 1996), allowing for a blue/white selection of cells containing vector with inserts. White cells were isolated and used to obtain plasmid DNA for sequencing.

cDNA Library from Human Neonatal Fibroblasts (HNFF)

Human neonatal fibroblast cells were grown in culture from explants of human neonatal foreskin and the total RNA extracted from these cells using established protocols. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library was then prepared from the mRNA by reverse transcriptase synthesis using a Lambda ZAP cDNA library synthesis kit (Stratagene).

cDNA Library from Mouse Embryonic Skin (MEMS)

Embryonic skin was micro-dissected from day 13 post coitum Balb/c mice. Embryonic skin was washed in phosphate buffered saline and mRNA directly isolated from the tissue using the Quick Prep Micro mRNA purification kit (Pharmacia, Sweden). The mRNA was then used to prepare cDNA libraries as described above for the DEPA library.

cDNA Library from Mouse Stem Cells (KSCL) and Transit Amplifying (TRAM) Cells

Pelts obtained from 1-2 day post-partum neonatal Balb/c mice were washed and incubated in trypsin (BRL Life Technologies) to separate the epidermis from the dermis. Epidermal tissue was disrupted to disperse cells, which were then resuspended in growth medium and centrifuged over Percoll density gradients prepared according to the manufacturer's protocol (Pharmacia, Sweden). Pelleted cells were labeled using Rhodamine 123 (Bertoncello I, Hodgson GS and Bradley TR, Exp Hematol. 13:999-1006, 1985), and analyzed by flow cytometry (Epics Elite Coulter Cytometry, Hialeah, Fla). Single cell suspensions of rhodamine-labeled murine keratinocytes were then labeled with a cross reactive anti-rat CD29 biotin monoclonal antibody (Pharmingen, San Diego, Calif.; clone Ha2/5). Cells were washed and incubated with anti-mouse CD45 phycoerythrin conjugated monoclonal antibody (Pharmingen; clone 30F11.1, 10 ug/ml) followed by labeling with streptavidin spectral red (Southern Biotechnology, Birmingham, Ala.). Sort gates were defined using listmode data to identify four populations: CD29 bright rhodamine dull CD45 negative cells; CD29 bright rhodamine bright CD45 negative cells; CD29 dull rhodamine bright CD45 negative cells; and CD29 dull rhodamine dull CD45 negative cells. Cells were sorted, pelleted and snap frozen prior to storage at −80° C. This protocol was followed multiple times to obtain sufficient cell numbers of each population to prepare cDNA libraries. Skin stem cells and transit amplifying cells are known to express CD29, the integrin β1 chain. CD45, a leukocyte specific antigen, was used as a marker for cells to be excluded in the isolation of skin stem cells and transit amplifying cells. Keratinocyte stem cells expel the rhodamine dye more efficiently than transit amplifying cells. The CD29 bright, rhodamine dull, CD45 negative population (putative keratinocyte stem cells; referred to as KSCL), and the CD29 bright, rhodamine bright, CD45 negative population (keratinocyte transit amplifying cells; referred to as TRAM) were sorted and mRNA was directly isolated from each cell population using the Quick Prep Micro mRNA purification kit (Pharmacia, Sweden). The mRNA was then used to prepare cDNA libraries as described above for the DEPA library.

cDNA Library from Epithelial Cells (MFSE)

Skin epidermis was removed from flaky skin fsn -/- mice (The Jackson Laboratory, Bar Harbour, Me.), the cells dissociated and the resulting single cell suspension placed in culture. After four passages, the cells were harvested. Total RNA, isolated using TRIzol Reagent (BRL Life Technologies, Gaithersburg, Md.), was used to obtain mRNA using a Poly(A)Quik mRNA isolation kit (Stratagene, La Jolla, Calif.), according to the manufacturer's specifications. A cDNA expression library (referred to as the MFSE library) was then prepared from the mRNA by Reverse Transcriptase synthesis using a Lambda ZAP Express cDNA library synthesis kit (Stratagene, La Jolla, Calif.).

cDNA Libraries from Human Small Airway Epithelial Cells (HLEA and HLEB)

Human small airway epithelium cells SAEC (Cell line number CC-2547, Clonetics Normal Human Cell Systems, Cambrex Corporation, East Rutherford N.J.) transformed with human papilloma virus E6E7 that was infected with the bacterium Yersinia enterocolitica (ATCC No. 51871, American Type Culture Collection, Manassas Va.) and the long form of the Respiratory Syncytial Virus (RSV, ATCC No. VR26), were used as source of RNA to construct the libraries called HLEA and HLEB. Cells from the twelfth passage of SAEC cells were infected with Y. enterocolitica for 2 hours at an initial seed of 12.5 bacteria per cell. The cells were disinfected with gentamycin (100 μg/ml) for 2 hours and harvested 4 hours after infection. The cells were then infected with RSV at a moiety of infection of 0.7 for 1 hour and incubated for 6 and 24 hours. Cells were harvested and the RNA extracted following standard protocols.

Total RNA, isolated using TRIzo1 Reagent (BRL Life Technologies, Gaithersburg, Md.), was used to obtain mRNA using a Poly(A) Quik mRNA isolation kit (Stratagene, La Jolla, Calif.), according to the manufacturer's specifications. Two cDNA expression libraries were then prepared from the mRNA by reverse transcriptase synthesis using a Lambda ZAP cDNA library synthesis kit (Stratagene).

cDNA Library from Epithelial Cells (HNKA)

The subtracted cDNA library (HNKA) from human natural killer (NK) cells was constructed as follows. A NK library was first constructed using pooled RNA extracted from primary NK cells from multiple donors, stimulated for 4 or 20 hours with IL-2 (10 ng/ml), IL-12 (1 ng/ml), IL-15 (50 ng/ml), interferon alpha (IFN-a; 1,000 U/ml) immobilized anti-CD16 or immobilized anti-NAIL antibody, or from unstimulated cells. RNA was extracted following standard procedures. cDNA was prepared using a TimeSaver kit (Pharmacia, Uppsala, Sweden) following the manufacturer's protocol. The cDNA was ligated to BglII adaptors and size-selected using cDNA sizing columns (Gibco BRL, Gaithersburg Md.). The size-selected NK cDNA was ligated into a pDc 409 vector and transformed into E. coli DH105 cells. Single-stranded DNA was prepared from the plasmid library using a helper phage (Stratagene).

A second cDNA library (referred to as FF cDNA library) was constructed using fetal foreskin tissue. RNA was extracted and cDNA prepared following standard protocols. The cDNA was ligated into the plasmid pBluescript following standard protocols. 10 μg of the FF cDNA library was linearized with the restriction endonuclease NotI and used as template to synthesize biotin-labeled cRNA using SP6 polymerase.

The subtracted NK cell library (HNKA) was constructed as follows. The biotinylated FF cRNA was mixed with the NK library, ethanol precipitated and resuspended in 5 μl buffer (50 mM HEPES pH 7.4, 10 mM EDTA, 1.5 MNaCl, 0.2% SDS). After addition of 5 μl formamide and heating to 95° for 1 min, the material was left to hybridize for 24 hours at 42° C. 90 μl of 10 mM HEPES pH 7.3, 1 mM EDTA and 15 μl streptavidin was added followed by an incubation for 20 min at 50° C. This step was repeated again after extraction with phenol/chloroform.

To the final extracted aqueous phase, the following were added: NaCl to 0.2 M, 1 μl glycogen and 2 volumes of ethanol. After an overnight precipitation at −20° C., the DNA was pelleted and resuspended in 10 μl water. A second round of subtraction was performed as above and the DNA transformed into E. coli DH105.

cDNA sequences were obtained by high-throughput sequencing of the cDNA libraries described above using a Perkin Elmer/Applied Biosystems Division Prism 377 sequencer.

### EXAMPLE 2

### Characterization of Isolated cDNA Sequences

The isolated cDNA sequences were compared to sequences in the EMBL DNA database using the computer algorithms FASTA and/or BLASTN. The corresponding protein sequences (DNA translated to protein in each of 6 reading frames) were compared to sequences in the SwissProt database using the computer algorithms FASTX and/or BLASTX. Comparisons of DNA sequences provided in SEQ ID NO: 1-119 to sequences in the EMBL DNA database (using FASTA) and amino acid sequences provided in SEQ ID NO: 120-197 to sequences in the SwissProt database (using FASTX) were made as of Mar. 21, 1998. Comparisons of DNA sequences provided in SEQ ID NO: 198-274 to sequences in the EMBL DNA database (using BLASTN) and amino acid sequences provided in SEQ ID NO: 275-348 to sequences in the SwissProt database (using BLASTP) were made as of Oct. 7, 1998. Comparisons of DNA sequences provided in SEQ ID NO: 349-372 to sequences in the EMBL DNA database (using BLASTN) and amino acid sequences provided in SEQ ID NO: 373-398 to sequences in the SwissProt database (using BLASTP) were made as of Jan. 23, 1999. Comparisons of polynucleotide sequences provided in SEQ ID NO: 418-455 and 466-487 to sequences in the EMBL DNA database (using BLASTN) and polypeptide sequences provided in SEQ ID NO: 456-463 and 488-509 to sequences in the SwissProt database (using BLASTP) were made as of Apr. 23, 2000. Comparisons of polynucleotide sequences provided in SEQ ID NO: 510 and 511 to sequences in the EMBL DNA database (using BLASTN) and polypeptide sequences provided in SEQ ID NO: 512 and 513 to sequences in the SwissProt database (using BLASTP) were made as of Jul. 11, 2000. Comparisons of polynucleotide sequences provided in SEQ ID NO: 514-623 to sequences in the EMBL66-HTGs +ENSEMBL (May 1, 2001) DNA database (using BLASTN) and polypeptide sequences provided in SEQ ID NO: 624-725 to sequences in the SP_TR_NRDB+ENSEMBL (Apr. 30, 2001) database (using BLASTP) were made as of May 16, 2001.

Isolated cDNA sequences and their corresponding polypeptide sequences were computer analyzed for the presence of signal sequences identifying secreted molecules. Isolated cDNA sequences that have a signal sequence at a putative start site within the sequence are provided in SEQ ID NO: 1-44, 198-238, 349-358, 399, 418-434, 440-449 and 466-471, 516, 519, 520, 523-527, 531, 532, 535-537, 548, 555, 574-580, 585-587, 589, 593, 595, 596, 598-601, 605-607, 609, 612, 613, 615, 616 and 622. The cDNA sequences of SEQ ID NO: 1-6, 198-199, 349-352, 354, 356-358,419-428, 430-433, 440-444, 446-448, 466, 468-470, 519, 520, 523, 524, 529, 531, 532, 535-537, 579, 585, 587, 598, 605, 609, 613 and 622 were determined to have less than 75% identity (determined as described above), to sequences in the EMBL database using the computer algorithms FASTA or BLASTN, as described above. The polypeptide sequences of SEQ ID NO: 120-125, 275-276, 373-380, 382, 456, 457, 460-462, 488-493, 633, 637, 642, 683, 685, 691, 693, 703, 706, 710, 714, 717, 718, 720, 721 and 725 were determined to have less than 75% identity (determined as described above) to sequences in the SwissProt database using the computer algorithms FASTX or BLASTP, as described above.

Further sequencing of some of the isolated partial cDNA sequences resulted in the isolation of the full-length cDNA sequences provided in SEQ ID NOS: 7-14, 200-231, 372, 418-422, 441-448, 514, 516, 557-561, 567, 568, 619 and 621. The polypeptide sequences encoded by the cDNA sequences of SEQ ID NO: 7-14, 200-231, 372, 514, 516, 557-561, 567, 568, 619 and 621 are provided in SEQ ID NOS: 126-133, 277-308, 396, 624, 626, 666-669, 674 and 724, respectively. The cDNA sequences of SEQ ID NO: 418-422 encode the same amino acid sequences as the cDNA sequences of SEQ ID NO: 7 and 11-14, namely SEQ ID NO: 126 and 130-133, respectively. Comparison of the full-length cDNA sequences with those in the EMBL database using the computer algorithm FASTA or BLASTN, as described above, revealed less than 75% identity (determined as described above) to known sequences, except for the polynucleotides in SEQ ID NOS: 516, 560 and 619. Comparison of the amino acid sequences provided in SEQ ID NOS: 126-133, 277-308, 666, 668, 669 and 724 with those in the SwissProt database using the computer algorithms FASTX or BLASTP, as described above, revealed less than 75% identity (determined as described above) to known sequences.

Comparison of the polypeptide sequences corresponding to the cDNA sequences of SEQ ID NOS: 15-23 with those in the HMBL database using the computer algorithm FASTA database showed less than 75% identity (determined as described above) to known sequences. These polypeptide sequences are provided in SEQ ID NOS: 134-142.

Further sequencing of some of the isolated partial cDNA sequences resulted in the isolation of full-length cDNA sequences provided in SEQ ID NOS: 24-44, 232-238, 423-434, 449, 466, 468-470, 475, 476 and 484. The polypeptide sequences encoded by the cDNA sequences of SEQ ID NO: 24-44, 232-238, 429, 466, 468-470, 475, 476 and 484 are provided in SEQ ID NOS: 143-163, 309-315, 456, 488, 490-492, 497, 498 and 506, respectively. The cDNA sequences of SEQ ID NO: 423-428, 430-434 and 449 encode the same polypeptide sequences as the cDNA sequences of SEQ ID NO: 27-29, 34, 35, 37, 40-44 and 238, namely SEQ ID NO: 146-148, 153, 154, 156, 159-163 and 315, respectively. These polypeptide sequences were determined to have less than 75% identity, determined as described above to known sequences in the SwissProt database using the computer algorithm FASTX.

Isolated cDNA sequences having less than 75% identity to known expressed sequence tags (ESTs) or to other DNA sequences in the public database, or whose corresponding polypeptide sequence showed less than 75% identity to known protein sequences, were computer analyzed for the presence of transmembrane domains coding for putative membrane-bound molecules. Isolated cDNA sequences that have one or more transmembrane domain(s) within the sequence are provided in SEQ ID NOS: 45-63, 239-253, 359-364, 400-402, 435, 436, 450-452, 455, 470-472, 542, 553-555, 573, 576, 581, 592, 593, 595 and 606. The cDNA sequences of SEQ ID NOS: 45-48, 239-249, 359-361, 363, 450, 451, 455, 472, 473, 553-555, 573, 576 and 592 were found to have less than 75% identity (determined as described above) to sequences in the EMBL database, using the PASTA or BLASTN computer algorithms. The polypeptide sequences encoded by the cDNA sequences of SEQ ID NO: 45-48, 239-249, 359-361, 363, 450, 451, 472, 473, 553-555, 573 and 606 (provided in SEQ ID NOS: 164-167, 316-326, 383, 385-388, 407-408, 460, 461, 494, 495, 662, 663, 664, 679, 682 and 711 respectively) were found to have less than 75% identity, determined as described above, to sequences in the SwissProt database using the FASTX or BLASTP database. The cDNA sequence of SEQ ID NO: 455 encodes the same polypeptide sequence as the cDNA sequence of SEQ ID NO: 359, namely SEQ ID NO: 383.

Comparison of the polypeptide sequences corresponding to the cDNA sequences of SEQ ID NOS: 49-63, 250-253, 436 and 452 with those in the SwissProt database showed less than 75% identity (determined as described above) to known sequences. These polypeptide sequences are provided in SEQ ID NOS: 168-182, 327-330, 457 and 462, respectively.

Using automated search programs to screen against sequences coding for molecules reported to be of therapeutic and/or diagnostic use, some of the cDNA sequences isolated as described above in Example 1 were determined to encode polypeptides that are family members of known protein families. A family member is herein defined to have at least 25% identity in the translated polypeptide to a known protein or member of a protein family. These cDNA sequences are provided in SEQ ID NOS: 64-76, 254-264, 365-369, 403, 437-439, 453, 454, 475-487, 510, 511, 514-527, 529-531, 533-536, 538-546, 548, 549, 553-559, 562, 564, 565, 567, 569-575, 577-589, 591-602, 604-612, 616-618, 621 and 622. The polypeptide sequences encoded by the cDNA sequences of SEQ ID NO: 64-76, 254-264, 365-369, 403, 438, 439, 453, 475-487, 510 and 511, 514-527, 529-531, 533-536, 538-546, 548, 549, 553-559, 562, 564, 565, 567, 569-575, 577-589, 591-602, 604-612, 616-618, 621 and 622 are provided in SEQ ID NOS: 183-195, 331-341, 389-393, 409, 458, 459, 463, 497-509, 624-637, 639-641, 643-646, 648-656, 658, 659, 662-668, 670, 672-681, 683-707, 709-717 and 721-725, respectively. The cDNA sequences of SEQ ID NO: 437 and 454 encode the same amino acid sequences as the cDNA sequences of SEQ ID NO: 68 and 262, namely SEQ ID NO: 187 and 339, respectively. The cDNA sequences of SEQ ID NOS: 64-68, 254-264, 365-369, 437-439, 453, 454, 475-478, 480-482, 484, 485, 487, 511, 514, 515, 517-520, 522, 523, 525, 529-531, 535, 536, 538, 541, 544-546, 549, 553-559, 564, 565, 567, 569-573, 579, 587, 588, 592, 597, 598, 602, 604, 605, 608-611, 617, 621 and 622 show less than 75% identity (determined as described above) to sequences in the EMBL database using the FASTA or BLASTN computer algorithms. Similarly, the amino acid sequences of SEQ ID NOS: 183-195, 331-341, 389-393, 458, 459, 463, 497, 498, 503-505, 507-509, 512, 513, 628, 632, 633, 637, 640, 655, 662-666, 668, 672, 673, 676, 679, 683, 685, 688, 691, 693, 694, 702, 703, 706, 707, 710, 711, 713, 714, 717, 721, 722 and 725 show less than 75% identity to sequences in the SwissProt database.

The isolated cDNA sequences encode proteins that influence the growth, differentiation and activation of several cell types, and that may usefully be developed as agents for the treatment and diagnosis of skin wounds, cancers, growth and developmental defects, and inflammatory disease. The utility for certain of the proteins of the present invention, based on similarity to known proteins, is provided in Table 2 below, together with the location of signal peptides and transmembrane domains for certain of the inventive sequences:

TABLE 2
FUNCTIONS OF NOVEL PROTEINS

| P/N | A/A | |
|---|---|---|
| SEQ | SEQ. | |
| ID NO: | ID NO. | SIMILARITY TO KNOWN PROTEINS; FUNCTION |
| 64, | 183, | Slit, a secreted molecule required for central nervous |
| 372 | 396 | system development |
| 65 | 184 | Immunoglobulin receptor family. About 40% of |
| | | leukocyte membrane polypeptides contain |
| | | immunoglobulin superfamily domains |
| 66, | 185, | RIP protein kinase, a serine/threonine kinase that |
| 403, | 409, | contains a death domain to mediate apoptosis |
| 510 | 512 | |
| 67 | 186 | Extracellular protein with epidermal growth factor |
| | | domain capable of stimulating fibroblast proliferation |
| 68, | 187 | Transforming growth factor alpha, a protein which |
| 437 | | binds epidermal growth factor receptor and stimulates |
| | | growth and mobility of keratinocytes |
| 69 | 188 | DRS protein which has a secretion signal component |
| | | and whose expression is suppressed in cells trans- |
| | | formed by oncogenes |
| 70 | 189 | A33 receptor with immunoglobulin-like domains and |
| | | is expressed in greater than 95% of colon tumors |
| 71 | 190 | Interleukin-12 alpha subunit, component of a cytokine |
| | | that is important in the immune defense against intra- |
| | | cellular pathogens. IL-12 also stimulates proliferation |
| | | and differentiation of TH1 subset of lymphocytes |
| 72 | 191 | Tumor Necrosis Factor receptor family of proteins that |
| | | are involved in the proliferation, differentiation and |
| | | death of many cell types including B and T |
| | | lymphocytes. |
| 73 | 192 | Epidermal growth factor family proteins which stimu- |
| | | late growth and mobility of keratinocytes and epithelial |
| | | cells. EGF is involved in wound healing. It also |
| | | inhibits gastric acid secretion. |
| 74 | 193 | Fibronectin Type III receptor family. The fibronectin |
| | | III domains are found on the extracellular regions of |
| | | cytokine receptors |
| 75 | 194 | Serine/threonine kinases (STK2_HUMAN) which |
| | | participate in cell cycle progression and signal trans- |
| | | duction |
| 76 | 195 | Immunoglobulin receptor family |
| 254 | 331 | Receptor with immunoglobulin-like domains and |
| | | homology to A33 receptor which is expressed in |
| | | greater than 95% of colon tumors |
| 255 | 332 | Epidermal growth factor family proteins which stimu- |
| | | late growth and mobility of keratinocytes and epithelial |
| | | cells. EGF is involved in wound healing. It also |
| | | inhibits gastric acid secretion. |
| 256 | 333 | Serine/threonine kinases (STK2_HUMAN) which |
| | | participate in cell cycle progression and signal trans- |
| | | duction |
| 257 | 334 | Contains protein kinase and ankyrin domains. Possible |
| | | role in cellular growth and differentiation. |
| 258 | 335 | Notch family proteins which are receptors involved in |
| | | cellular differentiation. |
| 259 | 336 | Extracellular protein with epidermal growth factor |
| | | domain capable of stimulating fibroblast proliferation. |
| 260, | 337, | Fibronectin Type III receptor family. The fibronectin |
| 453 | 463 | III domains are found on the extracellular regions of |
| | | cytokine receptors. |
| 261 | 338 | Immunoglobulin receptor family |
| 262 | 339 | ADP/ATP transporter family member containing a |
| | | calcium binding site. |
| 263 | 340 | Mouse CXC chemokine family members are regulators |
| | | of epithelial, lymphoid, myeloid, stromal and neuronal |
| | | cell migration and cancers, agents for the healing of |
| | | cancers, neuro-degenerative diseases, wound healing, |
| | | inflammatory autoimmune diseases like psoriasis, |
| | | asthma, Crohns disease and as agents for the preven- |
| | | tion of HIV-1 of leukocytes |
| 264 | 341 | Nucleotide-sugar transporter family member. |
| 365 | 389 | Transforming growth factor betas (TGF-betas) are |
| | | secreted covalently linked to latent TGF-beta-binding |
| | | proteins (LTBPs). LTBPs are deposited in the extra- |
| | | cellular matrix and play a role in cell growth or |
| | | differentiation. |
| 366 | 390 | Integrins are Type I membrane proteins that function |
| | | as laminin and collagen receptors and play a role in |
| | | cell adhesion. |
| 367 | 391 | Integrins are Type I membrane proteins that function |
| | | as laminin and collagen receptors and play a role in |
| | | cell adhesion. |
| 368 | 392 | Cell wall protein precursor. Are involved in cellular |
| | | growth or differentiation. |
| 369 | 393 | HT protein is a secreted glycoprotein with an EGF-like |
| | | domain. It functions as a modulator of cell growth, |
| | | death or differentiation. |
| 467 | 489 | Myb proto-oncogene (c-Myb), involved in transcrip- |
| | | tion regulation and activation of transcription |
| 471 | 493 | Chondroitin sulfotransferase, a member of the HNK-1 |
| | | sulfotransferase family. These molecules are involved |
| | | in the pathogenesis of arteriosclerosis, and prolifera- |
| | | tion of arterial smooth muscle cells during develop- |
| | | ment of arteriosclerosis. |
| 472 | 494 | 36 kDa nucleolar protein HNP36, a novel growth |
| | | factor responsive gene expressed in the pituitary and |
| | | parathyroid glands |
| 475 | 497 | Zinc protease is a matrix metalloproteinase whose |
| | | activity is directed against components of the extra- |
| | | cellular matrix and play an important role in the |
| | | growth, metastasis and angiogenesis of tumors. |
| 476 | 498 | Diapophytoene dehydrogenase crtn-like molecule. This |
| | | molecule is similar to the diapophytoene dehydro- |
| | | genase crt molecule in a major photosynthesis gene |
| | | cluster from the bacterium Heliobacillus mobilis |
| 477 | 499 | Protocadherin 3 family member, involved in cell to |
| | | cell interactions. |
| 478 | 500 | Integrins are Type I membrane proteins that function |
| | | as laminin and collagen receptors and play a role in |
| | | cell adhesion. |
| 479 | 501 | Integrin family member. Integrins are Type I |
| | | membrane proteins that function as laminin and |
| | | collagen receptors and play a role in cell adhesion. |
| 480 | 502 | Similar to secreted HT Protein, a secreted glycoprotein |
| | | with an EGF-like domain. It functions as a modulator |
| | | of cell growth, death or differentiation. |
| 481 | 503 | Agrin family member: Agrin is produced by moto- |
| | | neurons and induces the aggregation of nicotinic |
| | | acetylcholine receptors. |
| 482 | 504 | Macrophage Scavenger Receptors bind to a variety of |
| | | polyanionic ligands and display complex binding |
| | | characteristics. They have been implicated in various |
| | | macrophage-associated processes, including |
| | | atherosclerosis. |
| 483 | 505 | Similar to GARP, a member of the family of leucine- |
| | | rich repeat-containing proteins involved in platelet- |
| | | endothelium interactions. |
| 484 | 506 | Epidermal growth factor family proteins which stimu- |
| | | late growth and mobility of keratinocytes and epithelial |
| | | cells. EGF is involved in wound healing. It also |
| | | inhibits gastric acid secretion. |
| 485 | 507 | Colony stimulating growth factor family. |
| 486 | 508 | Cytokine receptors |
| 487 | 509 | IL17 Receptor to Interleukin 17 (IL17), a T cell |
| | | derived cytokine that may play a role in initiation or |
| | | maintenance of the inflammatory response. |
| 438 | 458 | MEGF6, a protein containing multiple EGF-like- |
| | | domains. |
| 439 | 459 | Protein kinase family member involved in signal |
| | | transduction. |
| 454 | | Peroxisomal calcium-dependent solute carrier, a new |
| | | member of the mitochondrial transporter superfamily. |
| 511 | 513 | Serine/threonine kinase NEK1 is a NIMA-related |
| | | protein kinase that phosphorylates serines and |
| | | threonines, but also possesses tyrosine kinase activity. |
| | | NEK1 has been implicated in the control of meiosis |
| | | and belongs to the NIMA kinase subfamily. |
| 514 | 624 | ₆₂₆Homologue isolated from rat dermal papilla of |
| | | integrin alpha-11/beta-1 that is involved in muscle |
| | | development and maintaining integrity of adult muscle |
| | | and other adult tissues. Integrin alpha-11/beta-1 is a |
| | | receptor for collagen and belongs to the integrin alpha |
| | | chain family. |
| 516 | 625 | This is a secreted molecule isolated from rat dermal |
| | | papillae with a signal peptide at the N-terminus (amino |
| | | acid residues 1 to 21; nucleotides 42 to 104). |
| 517 | 626 | Homologue isolated from a rat dermal papilla library |
| | | of OASIS (old astrocyte specifically-induced |
| | | substance) and that plays a role in regulation of the |
| | | response of astrocytes to inflammation and trauma of |
| | | the central nervous system (CNS) during gliosis. The |
| | | OASIS gene encodes a putative transcription factor |
| | | belonging to the cyclic AMP responsive element |
| | | binding protein/activating transcription factor (CREB/ |
| | | ATF) gene family (Honma et al., Brain Res. Mol. |
| | | Brain Res. 69: 93-103, 1999). |
| 519 | 628 | This is a secreted molecule isolated from rat dermal |
| | | papillae with a signal peptide at the N-terminus (amino |
| | | acid residues 1 to 24; nucleotides 50 to 121). |
| 520 | 630 | This is a secreted molecule isolated from rat dermal |
| | | papillae with a signal peptide at the N-terminus (amino |
| | | acid residues 1 to 35; nucleotides 67 to 171). |
| 523 | 633 | This is a secreted molecule isolated from rat dermal |
| | | papillae with a signal peptide at the N-terminus (amino |
| | | acid residues 1 to 17; nucleotides 3 to 53). |
| 524 | 634 | This is a secreted molecule isolated from rat dermal |
| | | papillae with a signal peptide at the N-terminus (amino |
| | | acid residues 1 to 20; nucleotides 13 to 72). |
| 525, | 635, | Homologue isolated from a rat dermal papilla library |
| 534 | 644 | of leucyl-specific aminopeptidase, PILS-AP and that |
| | | plays role in many physiological processes as a |
| | | substrate-specific peptidase. PILS is a new member of |
| | | the M1 famile of Zn-dependent aminopeptidases that |
| | | comprises members of closely related enzymes which |
| | | are known to be involved in a variety of physiologi- |
| | | cally important processes. |
| 526 | 636 | This is a secreted molecule isolated from rat dermal |
| | | papillae with a signal peptide at the N-terminus (amino |
| | | acid residues 1 to 26; nucleotides 114 to 191). |
| 527 | 637 | This is a secreted molecule isolated from rat dermal |
| | | papillae with a signal peptide at the N-terminus (amino |
| | | acid residues 1 to 26; nucleotides 23 to 100). |
| 529 | 639 | This is a secreted molecule isolated from rat dermal |
| | | papillae with a signal peptide at the N-terminus (amino |
| | | acid residues 1 to 17; nucleotides 37 to 87). |
| 530 | 640 | This is a homologue isolated from a rat dermal papilla |
| | | library of a maturase that is involved in RNA splicing. |
| 531 | 641 | This is a secreted molecule isolated from rat dermal |
| | | papillae with a signal peptide at the N-terminus (amino |
| | | acid residues 1 to 17; nucleotides 180 to 230). |
| 532 | 642 | This is a secreted molecule isolated from rat dermal |
| | | papillae with a signal peptide at the N-terminus (amino |
| | | acid residues 1 to 32; nucleotides 245 to 340). |
| 535 | 645 | This is a secreted molecule isolated from rat dermal |
| | | papillae with a signal peptide at the N-terminus (amino |
| | | acid residues 1 to 25; nucleotides 188 to 333). |
| 536 | 646 | This is a secreted molecule isolated from rat dermal |
| | | papillae with a signal peptide at the N-terminus (amino |
| | | acid residues 1 to 21; nucleotides 185 to 247). |
| 537 | 647 | This is a secreted molecule isolated from rat dermal |
| | | papillae with a signal peptide at the N-terminus (amino |
| | | acid residues 1 to 24; nucleotides 129 to 200). |
| 541 | 651 | This is a homologue isolated from a rat dermal papilla |
| | | library of a hepatoma-derived growth factor (HDGF) |
| | | that is involved in stimulation of cell proliferation. |
| 542 | 652 | This is a receptor-like molecule isolated from rat |
| | | dermal papillae with two transmembrane domains |
| | | (amino acid residues 20 to 40 and 58 to 78. |
| 545 | 655 | This is a homologue isolated from a rat dermal papilla |
| | | library of Link protein (LP) and that is involved in |
| | | bone formation. LP plays an essential role in |
| | | endochondral bone formation by stabilizing the supra- |
| | | molecular assemblies of aggrecan and hyaluronan |
| | | (Deak et al., Cytogenet. Cell Genet. 87: 75-79, 1999). |
| 548 | 658 | This is a homologue isolated from a rat dermal papilla |
| | | library of thrombospondin (TSP). It is a secreted |
| | | protein with a signal peptide in amino acid residues 1 |
| | | to 18 (nucleotides 210 to 263). TSP is an extracellular |
| | | matrix glycoprotein whose expression has been |
| | | associated with a variety of cellular processes includ- |
| | | ing growth and embryogenesis (Laherty et al., J. Biol. |
| | | Chem. 267: 3,274-3,281, 1992). |
| 553 | 662 | This is a receptor-like molecule isolated from rat |
| | | dermal papillae with a transmembrane domain (amino |
| | | acid residues 434 to 454. |
| 554 | 663 | This is a receptor-like molecule isolated from rat |
| | | dermal papillae with a transmembrane domain (amino |
| | | acid residues 546 to 566. |
| 555 | 664 | This is a homologue isolated from a rat dermal papilla |
| | | library of B7-like mouse GL50 (mGL50). It is a |
| | | receptor-like molecule with a signal peptide in residues |
| | | 1 to 24 (nucleotides 149 to 220) and a transmembrane |
| | | domain in amino acid residues 262 to 282. GL50 is a |
| | | specific ligand for the ICOS receptor and this interac- |
| | | tion functions in lymphocyte costimulation (Ling et al., |
| | | J. Immunol. 164: 1,653-1,657, 2000). |
| 557, | 666, | These molecules are differentially expressed in stem |
| 558, | 667, | cells but not in mature keratinocytes and are involved |
| 561- | 670- | in developmental processes. They may be employed |
| 572 | 678 | for diagnosis of tumors with an immature phenotype. |
| 559 | 668 | This is a homologue isolated from a mouse stem cell |
| | | library of ABSENT IN MELANOMA 1 protein AIM1 |
| | | and that can be used for diagnosis of tumours with an |
| | | immature phenotype. AIM1 is a novel gene whose |
| | | expression is associated with the experimental |
| | | reversal of tumorigenicity of human malignant |
| | | melanoma and belongs to the betagamma-crystallin |
| | | superfamily (Ray et al., Proc. Natl. Acad. Sci. USA |
| | | 94: 3,229-3,234, 1997) |
| 560 | 669 | Homologue isolated from a mouse stem cell library of |
| | | endothelin-convertin enzyme 2 (ECE-2) and that can |
| | | be used for diagnosis of tumours with an immature |
| | | phenotype. Endothelins (ET) are a family of potent |
| | | vasoactive peptides that are produced from biologically |
| | | inactive intermediates, termed big endothelins, via a |
| | | proteolytic processing at Trp21-Val/Ile22. ECE-2, that |
| | | produces mature ET-1 from big ET-1 both in vitro and |
| | | in transfected cells. ECE-2 acts as an intracellular |
| | | enzyme responsible for the conversion of endogenous- |
| | | ly synthesized big ET-1 at the trans-Golgi network, |
| | | where the vesicular fluid is acidified (Emoto and |
| | | Yanagisawa, J. Biol. Chem. 270: 15,262-15,268, |
| | | 1995). |
| 573 | 679 | Mouse homologue of EGF-like molecule containing |
| | | mucin-like hormone receptor 2 (EMR2). The isolated |
| | | molecule contains three transmembrane regions: amino |
| | | acid residues 20 to 40, 66 to 86 and 92 to 112. The |
| | | epidermal growth factor (EGF)-TM7 proteins [EMR1 |
| | | and EMR2, F4/80, and CD97] constitute a recently |
| | | defined class B GPCR subfamily and are predominant- |
| | | ly expressed on leukocytes. These molecules possess |
| | | N-terminal EGF-like domains coupled to a seven-span |
| | | transmembrane (7TM) moiety via a mucin-like spacer |
| | | domain (Lin et al., Genomics 67: 188-200, 2000). |
| 574 | 680 | This is a murine secreted molecule with a signal |
| | | peptide at the N-terminus (amino acid residues 1 to 17; |
| | | nucleotides 238 to 288). |
| 575 | 681 | Mouse homologue of a glucocortocoid-inducible |
| | | protein GIS5 with a signal peptide at the N-terminus |
| | | (amino acid residues 1 to 17; nucleotides 56-106). |
| 576 | 682 | This is a murine surface receptor-like molecule with a |
| | | signal peptide at the N-terminus (amino acid residues 1 |
| | | to 17; nucleotides 1179 to 199) and a transmembrane |
| | | domain (amino acid residues 179 to 199). |
| 577 | 683 | This is a murine secreted molecule with a signal |
| | | peptide at the N-terminus (amino acid residues 1 to 16; |
| | | nucleotides 55 to 102). |
| 578 | 684 | This is a murine secreted molecule with a signal |
| | | peptide at the N-terminus (amino acid residues 1 to 22; |
| | | nucleotides 12 to 77). |
| 579 | 685 | This is a murine secreted molecule with a signal |
| | | peptide at the N-terminus (amino acid residues 1 to 17; |
| | | nucleotides 82 to 132). |
| 580 | 686 | This is a murine secreted molecule with a signal |
| | | peptide at the N-terminus (amino acid residues 1 to 20; |
| | | nucleotides 20 to 79). |
| 581 | 687 | This is a murine receptor-like molecule with |
| | | transmembrane domains at amino acid residues 50 to |
| | | 70; 84 to 104; 116 to 136 and 179 to 198. |
| 585 | 691 | This is a murine secreted molecule with a signal |
| | | peptide at the N-terminus (amino acid residues 1 to 20; |
| | | nucleotides 260 to 319). |
| 586 | 695 | This is a murine secreted molecule with a signal |
| | | peptide at the N-terminus (amino acid residues 1 to 22; |
| | | nucleotides 295 to 360). |
| 587 | 693 | This is a mouse homologue of serotransferrin, also |
| | | known as siderophilin or beta-1-metal binding |
| | | globulin) and that is involved in iron transport. This |
| | | homologue is a secreted molecule with a signal peptide |
| | | at the N-terminus (amino acid residues 1 to 19; |
| | | nucleotides 43 to 99). Transferrins are iron binding |
| | | transport proteins which can bind two atoms of ferric |
| | | iron in association with the binding of an anion, |
| | | usually bicarbonate. It is responsible for the transport |
| | | of iron from sites of absorption and heme degradation |
| | | to those of storage and utilization. Serum transferrin |
| | | may also have a further role in stimulating cell pro- |
| | | liferation. Transferrin belongs to the transferrin |
| | | family. |
| 589 | 695 | This is a murine secreted molecule with a signal |
| | | peptide at the N-terminus (amino acid residues 1 to 25; |
| | | nucleotides 1 to 75). |
| 592 | 697 | This is a murine receptor-like molecule with a |
| | | transmembrane domain in amino acid residues 52 to |
| | | 72. |
| 593 | 698 | Mouse homologue of channel inducing factor (CHIF) |
| | | that plays a role in ion transport. The mouse |
| | | homologue has a signal peptide at the N-terminus of |
| | | the predicted polypeptide (amino acid residues 1 to 20; |
| | | nucleotides 102 to 161) and a transmembrane domain |
| | | (amino acid residues 38 to 58). CHIF evokes a |
| | | potassium channel activity (Attali et al., Proc. Natl. |
| | | Acad. Sci. USA 92: 6092-6096, 1995). |
| 595 | 700 | Homologue of hyaluronan receptor LYVE-1 that plays |
| | | a role in hyalyronan uptake. This mouse homologue |
| | | has the characteristic signal peptide and trans- |
| | | membrane domain of a receptor. A signal peptide was |
| | | identified in the isolated molecule in amino acid |
| | | residues 1 to 18 (nucleotides 62 to 115) and the trans- |
| | | membrane domain in amino acid residues 233 to 253. |
| | | The extracellular matrix glycosaminoglycan |
| | | hyaluronan (HA) is an abundant component of skin |
| | | and mesenchymal tissues where it facilitates cell |
| | | migration during wound healing, inflammation, and |
| | | embryonic morphogenesis. Both during normal tissue |
| | | homeostasis and particularly after tissue injury, HA is |
| | | mobilized from these sites through lymphatic vessels |
| | | to the lymph nodes where it is degraded before |
| | | entering the circulation for rapid uptake by the liver. |
| | | LYVE-1 is a receptor for HA on the lymph vessel wall |
| | | and plays a role in the transport of HA from tissue to |
| | | lymph (Banerji et al., J. Cell Biol. 144: 789-801, |
| | | 1999). |
| 596 | 701 | This is a murine secreted molecule with a signal |
| | | peptide at the N-terminus (amino acid residues 1 to 21; |
| | | nucleotides 7 to 69). |
| 598 | 703 | Homologue of tumor-associated glycoprotein E4 |
| | | (TAA1 or TAGE4) that belongs to the immuno- |
| | | globulin superfamily. This molecule has a signal |
| | | peptide at the N-terminus (amino acid residues 1 to 24; |
| | | nucleotides 71 to 142) and is therefore a secreted |
| | | protein. |
| 599 | 704 | Homologue of the LUNX protein, also known as |
| | | nasopharyngeal carcinoma-related protein, tracheal |
| | | epithelium enriched protein or plunc, that is expressed |
| | | in epithelial cells in the airways. It has a signal peptide |
| | | at the N-terminus (amino acid residues 1 to 19; |
| | | nucleotides 39 to 95). Expression of LUNX is |
| | | restricted to the trachea, upper airway, nasopharyngeal |
| | | epithelium and salivary gland (Bingle and Bingle, |
| | | Biochim. Biophys. Acta 1493: 363-367, 2000). |
| 600 | 705 | This is a murine secreted molecule with a signal |
| | | peptide at the N-terminus (amino acid residues 1 to 23; |
| | | nucleotides 136 to 204. |
| 601 | 706 | Homologue of prenylcysteine lyase (EC 4.4.1.18) and |
| | | that is involved in degradation of prenylated proteins. |
| | | It has a signal peptide at the N-terminus (amino acid |
| | | residues 1 to 28; nucleotides 22 to 105). Prenylcysteine |
| | | lyase is a specific enzyme involved in the final step of |
| | | prenylcysteine metabolism in mammalian cells. The |
| | | enzyme does not require NADPH as cofactor for |
| | | prenylcysteine degradation, thus distinguishing it from |
| | | cytochrome P450- and flavin-containing mono- |
| | | oxygenases that catalyze S-oxidation of thioethers |
| | | (Zhang et al., J. Biol. Chem. 274: 35802-35808, |
| | | 1999). |
| 605 | 710 | Homologue of endoplasmin, endoplasmic reticulum |
| | | protein 99 (ERp99), 94 kDa glucose-regulated protein |
| | | (GRP94) and polymorphic tumor rejection antigen 1 |
| | | (gp96). The isolated molecule has a signal peptide at |
| | | the N-terminus (amino acid residue 1 to 21; |
| | | nucleotides 1867 to 206). ERp99 is an abundant, |
| | | conserved transmembrane glycoprotein of the |
| | | endoplasmic reticulum membrane and homologous to |
| | | the 90-kDa heat shock protein (hsp90) and the 94-kDa |
| | | glucose regulated protein (GRP94) (Mazzarella and |
| | | Green, J. Biol. Chem. 262: 8875-8883, 1987). |
| 606 | 711 | Homologue of PILRalpha, formerly known as |
| | | inhibitory receptor PIRIIalpha and that is involved in |
| | | signal transduction in various cellular processes. This |
| | | molecule contains a signal peptide at the N-terminal |
| | | end (amino acid residues 1-21 and nucleotides 47 to |
| | | 139) and a transmembrane domain at amino acid |
| | | residues 191 to 211. SHP-1-mediated dephosphoryla- |
| | | tion of protein tyrosine residues is central to the |
| | | regulation of several cell signaling pathways. |
| | | PILRalpha, a novel immunoreceptor tyrosine-based |
| | | inhibitory motif-bearing protein, recruits SHP-1 upon |
| | | tyrosine phosphorylation and is paired with the |
| | | truncated counterpart PILRbeta (Mousseau et al., J. |
| | | Biol. Chem. 275: 4467-4474, 2000). |
| 607 | 712 | This is a murine secreted molecule with a signal |
| | | peptide at the N-terminus (amino acid residues 1 to 18; |
| | | nucleotides 38 to 91. |
| 609 | 714 | Homologue of retinal short-chain dehydrogenase/ |
| | | reductase retSDR2 that plays a role on retinal metabo- |
| | | lism. It has a signal peptide at the N-terminus at amino |
| | | acid residues 1-29 (nucleotides 302 to 388). Retinol |
| | | dehydrogenases (RDH) catalyze the reduction of all- |
| | | trans-retinal to all-trans-retinol within the photo- |
| | | receptor outer segment in the regeneration of bleached |
| | | visual pigments (Haeseleer et al., J. Biol. Chem. 273: |
| | | 21790-21799, 1998) |
| 612 | 717 | This is a murine secreted molecule with a signal |
| | | peptide at the N-terminus (amino acid residues 1 to 22; |
| | | nucleotides 6 to 71. |
| 613 | 718 | This is a murine secreted molecule with a signal |
| | | peptide at the N-terminus (amino acid residues 1 to 25; |
| | | nucleotides 210 to 284. |
| 615 | 720 | This is a murine secreted molecule with a signal |
| | | peptide at the N-terminus (amino acid residues 1 to 16; |
| | | nucleotides 70 to 117. |
| 616 | 721 | This is a murine secreted molecule with a signal |
| | | peptide at the N-terminus (amino acid residues 1 to 18; |
| | | nucleotides 1 to 54. |

The locations of open reading frames (ORFs) within certain of the inventive cDNA sequences are shown in Table 3, below.

TABLE 3
LOCATION OF OPEN READING FRAMES

| SEQ ID NO | | SEQ ID NO |
|---|---|---|
| Polynucleotide | ORF | Polypeptide |
| 514 | 1-2,067 | 624 |
| 515 | 2-730 | 625 |
| 516 | 42-1,772 | 626 |
| 517 | 1-681 | 627 |
| 518 | 170-416 | 628 |
| 519 | 50-770 | 629 |
| 520 | 67-708 | 630 |
| 521 | 110-613 | 631 |
| 522 | 41-457 | 632 |
| 523 | 3-230 | 633 |
| 524 | 13-573 | 634 |
| 525 | 64-2,856 | 635 |
| 526 | 114-599 | 636 |
| 527 | 23-520 | 637 |
| 528 | 953-1,138 | 638 |
| 529 | 37-687 | 639 |
| 530 | 145-366 | 640 |
| 531 | 180-1,508 | 643 |
| 532 | 245-442 | 642 |
| 533 | 125-595 | 643 |
| 534 | 64-2,856 | 644 |
| 535 | 188-727 | 645 |
| 536 | 185-1,081 | 646 |
| 537 | 129-308 | 647 |
| 538 | 32-853 | 648 |
| 539 | 2-268 | 649 |
| 540 | 3-875 | 650 |
| 541 | 284-892 | 651 |
| 542 | 37-276 | 652 |
| 543 | 127-1,794 | 653 |
| 544 | 1-735 | 654 |
| 545 | 142-939 | 655 |
| 546 | 51-1,082 | 656 |
| 547 | 143-328 | 657 |
| 548 | 210-3,728 | 658 |
| 549 | 26-1,354 | 659 |
| 551 | 1,236-1,892 | 660 |
| 552 | 853-1,178 | 661 |
| 553 | 54-1,356 | 662 |
| 554 | 637-2,244 | 663 |
| 555 | 149-1,072 | 664 |
| 556 | 18-449 | 665 |
| 557 | 275-1,171 | 666 |
| 558 | 453-1,133 | 667 |
| 559 | 104-2,449 | 668 |
| 560 | 463-687 | 669 |
| 562 | 1-1,107 | 670 |
| 563 | 2-883 | 671 |
| 564 | 188-2,902 | 672 |
| 565 | 3-524 | 673 |
| 567 | 2,584-3,996 | 674 |
| 569 | 1-960 | 675 |
| 570 | 315-599 | 676 |
| 571 | 1-414 | 677 |
| 572 | 806-1,912 | 678 |
| 573 | 120-752- | 679 |
| 574 | 2381,359 | 680 |
| 575 | 56-1,456 | 681 |
| 576 | 13-645 | 682 |
| 577 | 55-1,323 | 683 |
| 578 | 12-698 | 684 |
| 579 | 82-810 | 685 |
| 580 | 20-586 | 686 |
| 581 | 65-808 | 687 |
| 582 | 369-761 | 688 |
| 583 | 1-769 | 689 |
| 584 | 164-1,321 | 690 |
| 585 | 260-1,489 | 691 |
| 586 | 295-1,131 | 692 |
| 587 | 43-2,136 | 693 |
| 588 | 1-1,203 | 694 |
| 589 | 1-525 | 695 |
| 591 | 1-584 | 696 |
| 592 | 1-522 | 697 |
| 593 | 102-368 | 698 |
| 594 | 1-517 | 699 |
| 595 | 62-1,018 | 700 |
| 596 | 7-282 | 701 |
| 597 | 1-736 | 702 |
| 598 | 71-1,297 | 703 |
| 599 | 39-875 | 704 |
| 600 | 136-930 | 705 |
| 601 | 22-1,539 | 706 |
| 602 | 69-521 | 707 |
| 603 | 104-448 | 708 |
| 604 | 1-399 | 709 |
| 605 | 3,068-5,476 | 710 |
| 606 | 47-721 | 711 |
| 607 | 38-439 | 712 |
| 608 | 1-1,656 | 713 |
| 609 | 302-1,327 | 714 |
| 610 | 845-1,447 | 715 |
| 611 | 975-1,375 | 716 |
| 612 | 6-272 | 717 |
| 613 | 210-464 | 718 |
| 614 | 462-869 | 719 |
| 615 | 70-459 | 720 |
| 616 | 1-1,107 | 721 |
| 617 | 1-349 | 722 |
| 618 | 93-528 | 723 |
| 621 | 380-1,033 | 724 |
| 622 | 43-2,115 | 725 |

The cDNA sequences of SEQ ID NO: 514, 515, 516, 557, 558, 559, 560, 561, 567, 568, 619 and 621 are extended sequences of SEQ ID NO: 479, 480, 353, 91, 108, 82, 92, 81, 105, 90, 362 and 360, respectively. SEQ ID NO: 516, 520, 521, 523, 525, 526, 529, 534-536, 541-543, 546, 548, 549, 557, 574, 575, 577-581, 584-587, 589, 593, 595, 596, 598-601, 605, 607, 609, 610, 614, 616 and 622 represent full-length cDNA sequences.

The polynucleotide sequences of SEQ ID NOS: 77-117, 265-267, 404-405 and 557-611 are differentially expressed in either keratinocyte stem cells (KSCL) or in transit amplified cells (TRAM) on the basis of the number of times these sequences exclusively appear in either one of the above two libraries; more than 9 times in one and none in the other (Audic S. and Clayerie J -M, Genome Research, 7:986-995, 1997). The sequences of SEQ ID NOS: 77-89, 265-267 and 365-369 were determined to have less than 75% identity to sequences in the EMBL database using the computer algorithm FASTA or BLASTN, as described above. The polypeptide sequences encoded by the cDNA sequences of SEQ ID NO: 77-117, 265-267, 404-405 and 557-611 are provided in SEQ ID NOS: 666-718. The amino acid sequences of SEQ ID NOS: 666, 668, 669, 671-673, 675, 676, 679, 682, 683, 685, 688, 690, 691, 693, 694, 702, 703, 706-708, 710, 711, 713 and 714 show less than 75% identity to sequences in the SwissProt database.

The polypeptides encoded by these polynucleotide sequences have utility as markers for identification and isolation of these cell types, and antibodies against these proteins may be usefully employed in the isolation and enrichment of these cells from complex mixtures of cells. Isolated polynucleotides and their corresponding proteins exclusive to the stem cell population can be used as drug targets to cause alterations in regulation of growth and differentiation of skin cells, or in gene targeting to transport specific therapeutic molecules to skin stem cells.

### EXAMPLE 3

### Isolation and Characterization of the Human Homolog of muTR1

The human homolog of muTR1 (SEQ ID NO: 68), obtained as described above in Example 1, was isolated by screening 50,000 pfu's of an oligo dT primed HeLa cell cDNA library. Plaque lifts, hybridization, and screening were performed using standard molecular biology techniques (Sambrook, J, Fritsch, E F and Maniatis, T, eds., Molecular Cloning. A Laboratory Manual, 2nd ed., Cold Spring Harbor Laboratory Press, Cold Spring Harbor: New York, 1989). The determined cDNA sequence of the isolated human homolog (huTR1) is provided in SEQ ID NO: 118, with the corresponding polypeptide sequence being provided in SEQ ID NO: 196. The library was screened using an [α ³²P]-dCTP labeled double stranded cDNA probe corresponding to nucleotides 1 to 459 of the coding region within SEQ ID NO: 118.

The polypeptide sequence of huTR1 has regions similar to Transforming Growth Factor-alpha, indicating that this protein functions like an epidermal growth factor (EGF). EGF family members exist in a functional form as small peptides. Alignment of the functional peptides of the EGF family with SEQ ID NO: 196 revealed that an internal segment of SEQ ID NO: 196 (amino acids 54-104) shows greater than 40% identity to the active peptides of EGF, TGF-alpha and Epiregulin. The active peptides of the EGF family are sufficient for activity and contain several conserved residues critical for the maintenance of this activity. These residues are retained in huTR1. The inventive EGF-like protein will serve to stimulate keratinocyte growth and motility, and to inhibit the growth of epithelial-derived cancer cells. This novel gene and its encoded protein may thus be used as agents for the healing of wounds and regulators of epithelial-derived cancers.

Analysis of RNA Transcripts by Northern Blotting

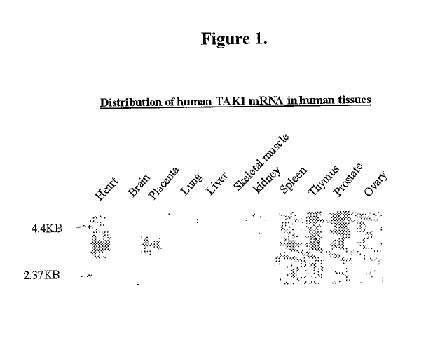

Northern analysis to determine the size and distribution of mRNA for huTR1 was performed by probing human tissue mRNA blots (Clontech) with a probe comprising nucleotides 93-673 of SEQ ID NO: 118, radioactively labeled with [α ³²P]-dCTP. Prehybridization, hybridization, washing and probe labeling were performed as described in Sambrook, et al., Ibid. mRNA for huTR1 was 3.5-4 kb in size and was observed to be most abundant in heart and placenta, with expression at lower levels being observed in spleen, thymus, prostate and ovary (FIG. 1).

The high abundance of mRNA for huTR1 in the heart and placenta indicates a role for huTR1 in the formation or maintenance of blood vessels, as heart and placental tissues have an increased abundance of blood vessels, and therefore endothelial cells, compared to other tissues in the body. This, in turn, demonstrates a role for huTR1 in angiogenesis and vascularization of tumors. This is supported by the ability of Transforming Growth Factor-alpha and EGF to induce de novo development of blood vessels (Schreiber, et al., Science 232:1250-1253, 1986) and stimulate DNA synthesis in endothelial cells (Schreiber, et al., Science 232:1250-1253, 1986), and their over-expression in a variety of human tumors.

Purification of muTR1 and huTR1

Polynucleotides 177-329 of muTR1 (SEQ ID NO: 268), encoding amino acids 53-103 of muTR1 (SEQ ID NO: 342), and polynucleotides 208-360 of huTR1 (SEQ ID NO: 269), encoding amino acids 54-104 of huTR1 (SEQ ID NO: 343), were cloned into the bacterial expression vector pProEX HT (BRL Life Technologies), which contains a bacterial leader sequence and N-terminal 6× Histidine tag. These constructs were transformed into competent XL1-Blue E. coli as described in Sambrook et al., Ibid.

Starter cultures of these recombinant XL1-Blue E.coli were grown overnight at 37° C. in Terrific broth containing 100 μg/ml ampicillin. This culture was spun down and used to inoculate 500 ml culture of Terrific broth containing 100 μg/ml ampicillin. Cultures were grown until the OD₅₉₅ of the cells was between 0.4 and 0.8, whereupon IPTG was added to 1 mM. Cells were induced overnight and bacteria were harvested by centrifugation.

Both the polypeptide of muTR1 (SEQ ID NO: 342; referred to as muTR1a) and that of huTR1 (SEQ ID NO: 343; referred to as huTR1a) were expressed in insoluble inclusion bodies. In order to purify the polypeptides muTR1a and huTR1a, bacterial cell pellets were re-suspended in lysis buffer (20 mM Tris-HCl pH 8.0, 10 mM beta mercaptoethanol, 1 mM PMSF). To the lysed cells, 1% NP40 was added and the mix incubated on ice for 10 minutes. Lysates were further disrupted by sonication on ice at 95W for 4×15 seconds and then centrifuged for 15 minutes at 14,000 rpm to pellet the inclusion bodies.

The resulting pellet was re-suspended in lysis buffer containing 0.5% w/v CHAPS and sonicated on ice for 5-10 seconds. This mix was stored on ice for 1 hour, centrifuged at 14,000 rpm for 15 minutes at 4° C. and the supernatant discarded. The pellet was once more re-suspended in lysis buffer containing 0.5% w/v CHAPS, sonicated, centrifuged and the supernatant removed as before. The pellet was re-suspended in solubilizing buffer (6 M Guanidine HCl, 0.5 M NaCl, 20 mM Tris HCl, pH 8.0), sonicated at 95 W for 4×15 seconds and then centrifuged for 20 minutes at 14,000 rpm and 4° C. to remove debris. The supernatant was stored at 4° C. until use.

Polypeptides muTR1a and huTR1a were purified by virtue of the N-terminal 6× Histidine tag contained within the bacterial leader sequence, using a Nickel-Chelating Sepharose column (Amersham Pharmacia, Uppsala, Sweden) and following the manufacturer's recommended protocol. In order to refold the proteins once purified, the protein solution was added to 5× its volume of refolding buffer (1 mM EDTA, 1.25 mM reduced glutathione, 0.25 mM oxidised glutathione, 20 mM Tris-HCl, pH 8.0) over a period of 1 hour at 4° C. The refolding buffer was stirred rapidly during this time, and stirring continued at 4° C. overnight. The refolded proteins were then concentrated by ultrafiltration using standard protocols.

Biological Activities of Polypeptides muTR1a and huTR1a

muTR1 and huTR1 are novel members of the EGF family, which includes EGF, TGFα, epiregulin and others. These growth factors are known to act as ligands for the EGF receptor. The pathway of EGF receptor activation is well documented. Upon binding of a ligand to the EGF receptor, a cascade of events follows, including the phosphorylation of proteins known as MAP kinases. The phosphorylation of MAP kinase can thus be used as a marker of EGF receptor activation. Monoclonal antibodies exist which recognize the phosphorylated forms of 2 MAP kinase proteins—ERK1 and ERK2.

In order to examine whether purified polypeptides of muTR1 a and huTR1 a act as a ligand for the EGF receptor, cells from the human epidermal carcinoma cell line A43 1 (American Type Culture Collection, No. CRL-1555, Manassas, Va.) were seeded into 6 well plates, serum starved for 24 hours, and then stimulated with purified muTR1 a or huTR1a for 5 minutes in serum free conditions. As a positive control, cells were stimulated in the same way with 10 to 100 ng/ml TGF-alpha or EGF. As a negative control, cells were stimulated with PBS containing varying amounts of LPS. Cells were immediately lysed and protein concentration of the lysates estimated by Bradford assay. 15 μg of protein from each sample was loaded onto 12% SDS-PAGE gels. The proteins were then transferred to PVDF membrane using standard techniques.

For Western blotting, membranes were incubated in blocking buffer (10 mM Tris-HCl, pH 7.6, 100 mM NaCl, 0.1% Tween-20, 5% non-fat milk) for 1 hour at room temperature. Rabbit anti-Active MAP kinase pAb (Promega, Madison, Wis.) was added to 50 ng/ml in blocking buffer and incubated overnight at 4° C. Membranes were washed for 30 mins in blocking buffer minus non-fat milk before being incubated with anti rabbit IgG-HRP antibody, at a 1:3500 dilution in blocking buffer, for 1 hour at room temperature. Membranes were washed for 30 minutes in blocking buffer minus non-fat milk, then once for 5 minutes in blocking buffer minus non-fat milk and 0.1% Tween-20. Membranes were then exposed to ECL reagents for 2 min, and then autoradiographed for 5 to 30 min.

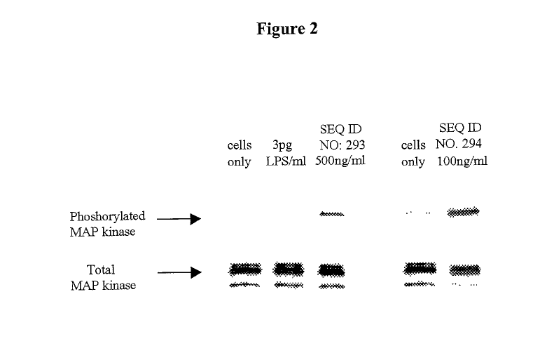

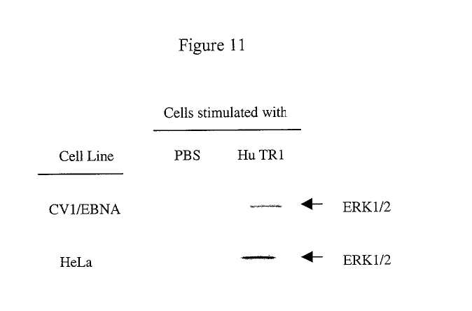

As shown in FIG. 2, both muTR1a and huTR1a were found to induce the phosphorylation of ERK1 and ERK2 over background levels, indicating that muTR1 and huTR1 act as ligands for a cell surface receptor that activates the MAP kinase signaling pathway, possibly the EGF receptor. As shown in FIG. 11, huTR1a was also demonstrated to induce the phosphorylation of ERK1 and ERK2 in CV1/EBNA kidney epithelial cells in culture, as compared with the negative control. These assays were conducted as described above. This indicates that huTR1a acts as a ligand for a cell surface receptor that activates the MAP kinase signaling pathway, possibly the EGF receptor in HeLa and CV1/EBNA cells.

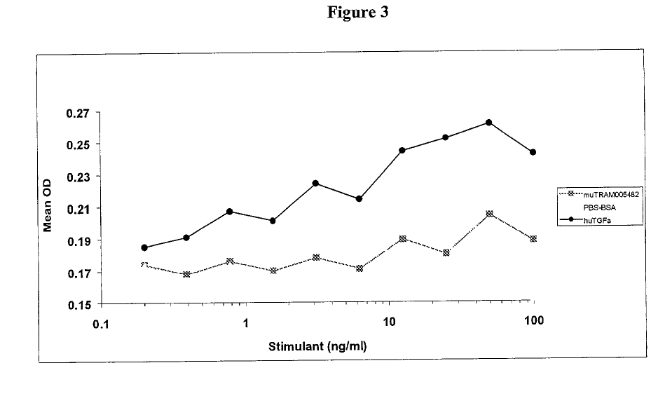

The ability of muTR1a to stimulate the growth of neonatal foreskin (NF) keratinocytes was determined as follows. NF keratinocytes derived from surgical discards were cultured in KSFM (BRL Life Technologies) supplemented with bovine pituatary extract (BPE) and epidermal growth factor (EGF). The assay was performed in 96 well flat-bottomed plates in 0.1 ml unsupplemented KSFM. MuTR1a, human transforming growth factor alpha (huTGFα) or PBS-BSA was titrated into the plates and 1×10³ NF keratinocytes were added to each well. The plates were incubated for 5 days in an atmosphere of 5% CO₂ at 37° C. The degree of cell growth was determined by MTT dye reduction as described previously (J. Imm. Meth. 93:157-165, 1986). As shown in FIG. 3, both muTR1 a and the positive control human TGFα stimulated the growth of NF keratinocytes, whereas the negative control, PBS-BSA, did not.

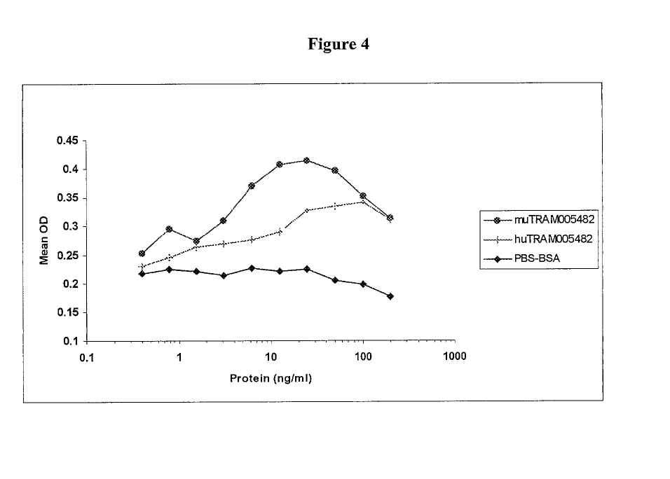

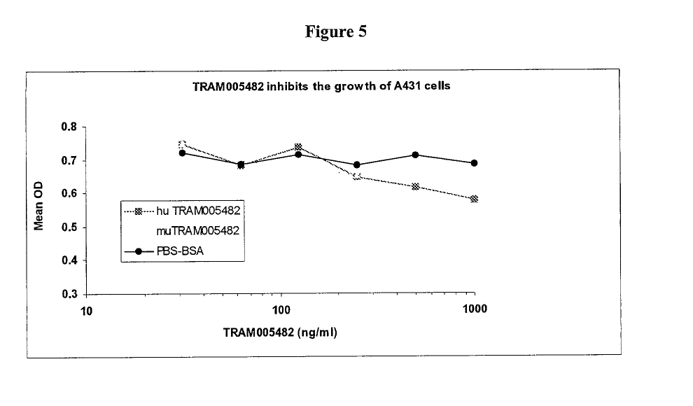

The ability of muTR1a and huTR1a to stimulate the growth of a transformed human keratinocyte cell line, HaCaT, was determined as follows. The assay was performed in 96 well flat-bottomed plates in 0.1 ml DMEM (BRL Life Technologies) supplemented with 0.2% FCS. MuTR1a, huTR1a and PBS-BSA were titrated into the plates and 1×10³ HaCaT cells were added to each well. The plates were incubated for 5 days in an atmosphere containing 10% CO₂ at 37° C. The degree of cell growth was determined by MTT dye reduction as described previously (J. Imm. Meth. 93:157-165, 1986). As shown in FIG. 4, both muTR1a and huTR1a stimulated the growth of HaCaT cells, whereas the negative control PBS-BSA did not.

The ability of muTR1a and huTR1a to inhibit the growth of A431 cells was determined as follows. Polypeptides muTR1a (SEQ ID NO: 342) and huTR1a (SEQ ID NO: 343) and PBS-BSA were titrated as described previously (J. Cell. Biol. 93:1-4, 1982), and cell death was determined using the MTT dye reduction as described previously (J. Imm. Meth. 93:157-165, 1986). Both muTR1a and huTR1a were found to inhibit the growth of A431 cells, whereas the negative control PBS-BSA did not (FIG. 5).

These results indicate that muTR1 and huTR1 stimulate keratinocyte growth and motility, inhibit the growth of epithelial-derived cancer cells, and play a role in angiogenesis and vascularization of tumors. This novel gene and its encoded protein may thus be developed as agents for the healing of wounds, angiogenesis and regulators of epithelial-derived cancers.

Upregulation of huTR1 and mRNA Expression

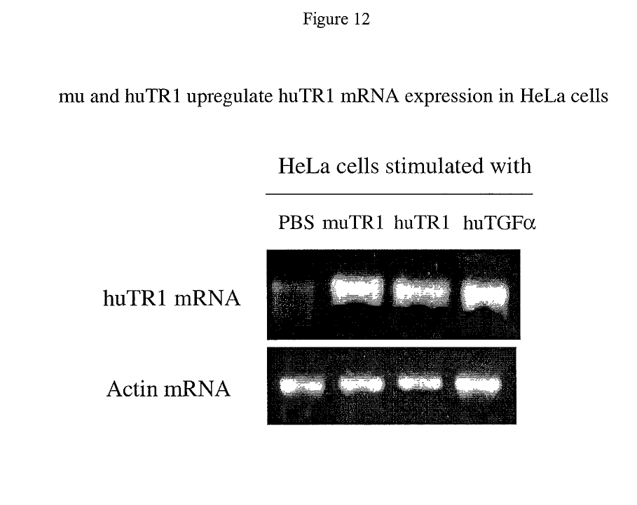

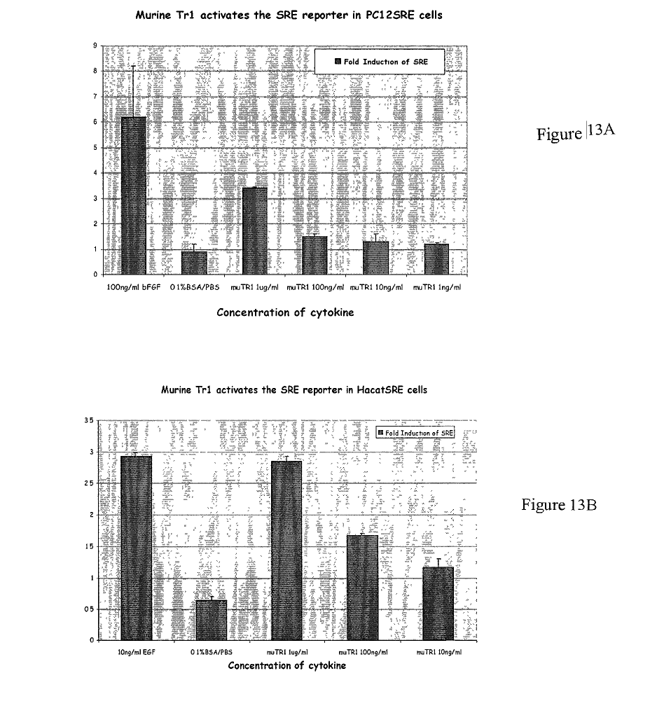

HeLa cells (human cervical adenocarcinoma) were seeded in 10 cm dishes at a concentration of 1×10⁶ cells per dish. After incubation overnight, media was removed and replaced with media containing 100 ng/ml of muTR1, huTR1, huTGFα, or PBS as a negative control. After 18 hours, media was removed and the cells lysed in 2 ml of TRIzol reagent (Gibco BRL Life Technologies, Gaithersburg, Md.). Total RNA was isolated according to the manufacturer's instructions. To identify mRNA levels of buTR1 from the cDNA samples, 1 μl of cDNA was used in a standard PCR reaction. After cycling for 30 cycles, 5 μl of each PCR reaction was removed and separated on a 1.5% agarose gel. Bands were visualized by ethidium bromide staining. As can be seen from FIG. 12, both mouse and human TR1 up-regulate the mRNA levels of huTR1 as compared with cells stimulated with the negative control of PBS. Furthermore, TGFa can also up-regulate the mRNA levels of huTR1.

These results indicate that TR1 is able to sustain its own mRNA expression and subsequent protein expression, and thus is expected to be able to contribute to the progression of diseases such as psoriasis where high levels of cytokine expression are involved in the pathology of the disease. Furthermore, since TGFα can up-regulate the expression of huTR1, the up-regulation of TR1 mRNA may be critical to the mode of action of TGFα.

Serum Response Element Reporter Gene Assay

The serum response element (SRE) is a promoter element required for the regulation of many cellular immediate-early genes by growth. Studies have demonstrated that the activity of the SRE can be regulated by the MAP kinase signaling pathway. Two cell lines, PC12 (rat pheochromocytoma—neural tumor) and HaCaT (human transformed keratinocytes), containing eight SRE upstream of an SV40 promotor and luciferase reporter gene were developed in-house. 5×10³ cells were aliquoted per well of 96 well plate and grown for 24 hours in their respective media. HaCaT SRE cells were grown in 5% fetal bovine serum (FBS) in D-MEM supplemented with 2 mM L-glutamine (Sigma, St. Louis, Mo.), 1 mM sodium pyruvate (BRL Life Technologies), 0.77 mM L-asparagine (Sigma), 0.2 mM arginine (Sigma), 160 mM penicillin G (Sigma), 70 mM dihydrostreptomycin (Roche Molecular Biochemicals, Basel, Switzerland), and 0.5 mg/ml geneticin (BRL Life Technologies). PC12 SRE cells were grown in 5% fetal bovine serum in Ham F12 media supplemented with 0.4 mg/ml geneticin (BRL Life Technologies). Media was then changed to 0.1% FBS and incubated for a further 24 hours. Cells were then stimulated with a titration of TR1 from 1 μg/ml. A single dose of basic fibroblast growth factor at 100 ng/ml (R&D Systems, Minneapolis, Minn.) or epidermal growth factor at 10 ng/ml (BRL Life Technologies) was used as a positive control. Cells were incubated in the presence of muTR1 or positive control for 6 hours, washed twice in PBS and lysed with 40 μl of lysis buffer (Promega). 10 μl was transferred to a 96 well plate and 10 μl of luciferase substrate (Promega) added by direct injection into each well by a Victor² fluorimeter (Wallac), the plate was shaken and the luminescence for each well read at 3×1 sec Intervals. Fold induction of SRE was calculated using the following equation: Fold induction of SRE=Mean relative luminescence of agonist/Mean relative luminescence of negative control.

As shown in FIG. 13, muTR1 activated the SRE in both PC-12 (FIG. 13A) and HaCaT (FIG. 13B) cells. This indicates that HaCaT and PC-12 cells are able to respond to muTR1 protein and elicit a response. In the case of HaCaT cells, this is a growth response. In the case of PC-12 cells, this may be a growth, a growth inhibition, differentiation, or migration response. Thus, TR1 may be important in the development of neural cells or their differentiation into specific neural subsets. TR1 may also be important in the development and progression of neural tumors.

Inhibition by the EGF Receptor Assay

The HaCaT growth assay was conducted as previously described, with the following modifications. Concurrently with the addition of EGF and TR1 to the media, anti-EGF Receptor (EGFR) antibody (Promega, Madison, Wis.) or the negative control antibody, mouse IgG (PharMingen, San Diego, Calif.), were added at a concentration of 62.5 ng/ml.

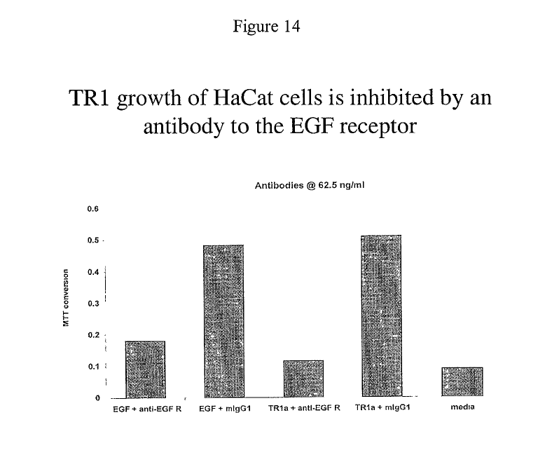

As seen in FIG. 14, an antibody which blocks the function of the EGFR inhibited the mitogenicity of TR1 on HaCaT cells. This indicates that the EGFR is crucial for transmission of the TR1 mitogenic signal on HaCaT cells. TR1 may bind directly to the EGF receptor. TR1 may also bind to any other members of the EGFR family (for example, ErbB-2, -3, and/or -4) that are capable of heterodimerizing with the EGFR.

Splice Variants of huTR1

A variant of huTR1 was isolated from the same library as huTR1, following the same protocols. The sequence referred to as huTR1-1 (also known as TR16) is a splice variant of huTR1 and consists of the ORF of huTR1 minus amino acids 15 to 44 and 87 to 137. These deletions have the effect of deleting part of the signal sequence and following amino terminal linker sequence, residues following the second cysteine residue of the EGF motif and the following transmembrane domain. However, cysteine residue 147 (huTR1 ORF numbering) may replace the deleted cysteine and thus the disulphide bridges are likely not affected. Therefore, huTR1-1 is an intracellular form of huTR1. It functions as an agonist or an antagonist to huTR1 or other EGF family members, including EGF and TGFa. The determined nucleotide sequence of huTR1-1, is given in SEQ ID NO: 412, with the corresponding amino acid sequence being provided in SEQ ID NO: 415.

Four additional splice variants of huTr1 were isolated by PCR on first strand cDNA made from RNA isolated from HeLa cells by standard protocols. These splice variants of huTR1 are referred to as TR1-2 (also known as TR1β), TR1-3 (also known as TR1γ), TR1ε and TR1φ.

TR1-2 consists of the ORF of huTR1 minus amino acids 95 to 137. This deletion has the effect of deleting the transmembrane domain. Therefore TR1-2 is a secreted form of huTR1 and binds with equal or greater affinity to the TR1 receptor as huTR1, since the EGF domain remains intact. It functions as an agonist or an antagonist to huTR1 or other EGF family members, including EGF and TGFα. The determined cDNA sequence of TR1-2 is given in SEQ ID NO: 410 and the corresponding amino acid sequence in SEQ ID NO: 413.

TR1-3 consists of the ORF of huTR1 minus amino acids 36 to 44 and amino acids 86 to 136. These deletions have the effect of deleting part of the amino terminal linker sequence, residues following the second cysteine of the EGF motif and the following transmembrane domain. However, cysteine residue 147 (huTR1 ORF numbering) may replace the deleted cysteine and thus the disulphide bridges are likely not affected. Therefore, TR1-3 is also a secreted form of huTR1 and functions as an agonist or an antagonist to huTR1 or other EGF family members, including EGF and TGFA. The determined cDNA sequence of TR1-3 is given in SEQ ID NO: 411 and the corresponding amino acid sequence is SEQ ID NO: 414.

TR1ε consists of the ORF of huTR1 minus amino acids 86 to 136. This deletion has the effect of deleting residues following the second cysteine of the EGF motif and the transmembrane domain. However, cysteine residue 147 (huTR1 ORF numbering) may replace the deleted cysteine and thus the disulphide bridges are likely not affected. Therefore, TR1 ε is also a secreted form of huTR1 and functions as an agonist or an antagonist to huTR1 or other EGF family members, including EGF and TGFA. The determined cDNA sequence of TR1ε is given in SEQ ID NO: 371 and the corresponding polypeptide sequence in SEQ ID NO: 395.

TR1φ consists of the ORF of huTR1 minus amino acids 36 to 44 and amino acids 95 to 136. These deletions have the effect of deleting part of the amino terminal linker sequence and the transmembrane domain. Therefore TR1φ is a secreted form of huTR1 and binds with equal or greater affinity to the TR1 receptor as huTR1, since the EGF domain remains intact. It functions as an agonist or an antagonist to huTR1 or other EGF family members, including EGF and TGFα. The determined nucleotide sequence of TR1φ is given in SEQ ID NO: 416 and the corresponding polypeptide sequence in SEQ ID NO: 417.

### EXAMPLE 4

### Identification, Isolation and Characterization of DP3

A partial cDNA fragment, referred to as DP3, was identified by differential display RT-PCR (modified from Liang P and Pardee AB, Science 257:967-971, 1992) using mRNA from cultured rat dermal papilla and footpad fibroblast cells, isolated by standard cell biology techniques. This double stranded cDNA was labeled with [α ³²P]-dCTP and used to identify a full length DP3 clone by screening 400,000 pfu's of an oligo dT-primed rat dermal papilla cDNA library. The determined full-length cDNA sequence for DP3 is provided in SEQ ID NO: 119, with the corresponding amino acid sequence being provided in SEQ ID NO: 197. Plaque lifts, hybridization and screening were performed using standard molecular biology techniques.

### EXAMPLE 5

### Isolation and Characterization of KS1

Analysis of RNA Transcripts by Northern Blotting

Northern analysis to determine the size and distribution of mRNA for muKS1 (SEQ ID NO: 263) was performed by probing murine tissue mRNA blots with a probe consisting of nucleotides 268-499 of muKS1, radioactively labeled with [α ³²P]-dCTP. Prehybridization, hybridization, washing, and probe labeling were performed as described in Sambrook, et al., Ibid. mRNA for muKS1 was 1.6 kb in size and was observed to be most abundant in brain, lung, or any muscle, and heart. Expression could also be detected in lower intestine, skin, bone marrow, and kidney. No detectable signal was found in testis, spleen, liver, thymus, stomach.

Human Homologue of muKS1

MuKS1 (SEQ ID NO: 263) was used to search the EMBL database (Release 50, plus updates to June, 1998) to identify human EST homologues. The top three homologies were to the following ESTs: accession numbers AA643952, HS1301003 and AA865643. These showed 92.63% identity over 285 nucleotides, 93.64% over 283 nucleotides and 94.035% over 285 nucleotides, respectively. Frame shifts were identified in AA643952 and HS1301003 when translated. Combination of all three ESTs identified huKS1 (SEQ ID NO: 270) and translated polypeptide SEQ ID NO: 344. Alignment of muKS1 and huKS1 polypeptides indicated 95% identity over 96 amino acids.

Identification of KSCL009274 cDNA Sequence

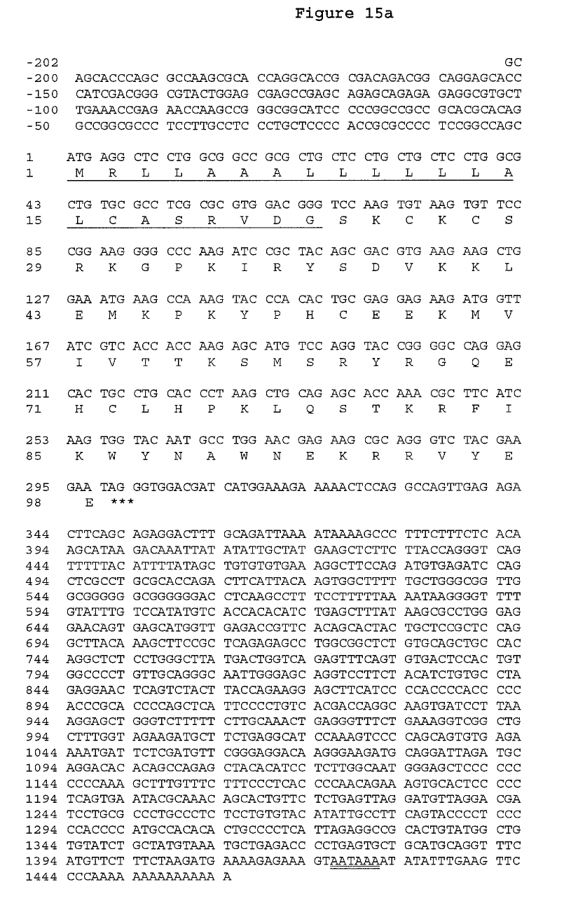

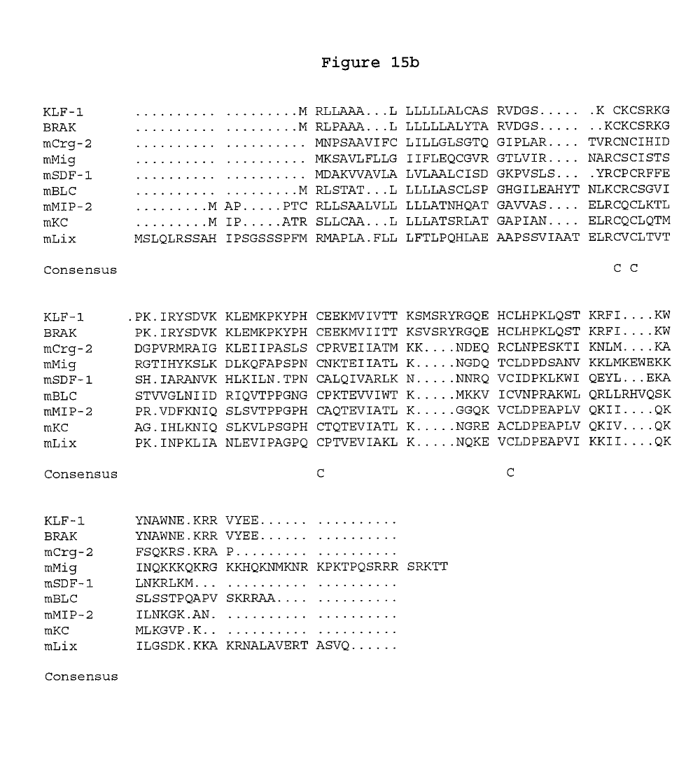

A directionally cloned cDNA library was constructed from immature murine keratinocytes and submitted for high-throughput sequencing. Sequence data from a clone designated KDCL009274 showed 35% identity over 72 amino acids with rat macrophage inflammatory protein-2B (MIP-2B) and 32% identity over 72 amino acids with its murine bomologue. The insert of 1633 bp (SEQ ID NO: 464; FIG. 15A) contained an open reading frame of 300 bp with a 5′ untranslated region of 202 bp and a 3′ untranslated region of 1161 bp. A poly-adenylation signal of AATAAA is present 19 base pairs upstream of the poly-A tail. The mature polypeptide (SEQ ID NO: 465) is 77 amino acids in length containing 4 conserved cysteines with no ELR motif. The putative signal peptide cleavage site beween GLY 22 and Ser 23 was predicted by the hydrophobicity profile. This putative chemokine was identical to KS 1. The full length sequence was screened against the EMBL database using the BLAST program and showed some identity at the nucleotide level with human EST clones AA643952, AA865643, and HS1301003, respectively. A recently described human CXC chemokine, BRAK, has some identity with KS 1 at the protein level. The alignment of KS 1 (referred to in FIG. 15B as KLF-1), BRAK, and other murine α-chemokines is shown in FIG. 15B. The phylogenetic relationship between KS1 and other α-chemokine family members was determiend using the Phylip program. KS1 and BRAK demonstrate a high degree of divergence from the other α-chemokines, supporting the relatively low homology shown in the multiple alignment.

Bacterial Expression and Purification of muKS1 and huKS1

Polynucleotides 269-502 of muKS1 (SEQ ID NO: 271), encoding amino acids 23-99 of polypeptide muKS1 (SEQ ID NO: 345), and polynucleotides 55-288 of huKS1 (SEQ ID NO: 272), encoding amino acids 19-95 of polypeptide huKS1 (SEQ ID NO: 346), were cloned into the bacterial expression vector pET-16b (Novagen, Madison, Wis.), which contains a bacterial leader sequence and N-terminal 6× Histidine tag. These constructs were transformed into competent XL1-Blue E.coli as described in Sambrook et al., Ibid.

Starter cultures of recombinant BL 21 (DE3) E.coli (Novagen) containing SEQ ID NO: 271 (muKS1a) and SEQ ID NO: 272 (huKS1a) were grown in NZY broth containing 100 μg/ml ampicillin (Gibco-BRL Life Technologies) at 37° C. Cultures were spun down and used to inoculate 800 ml of NZY broth and 100 μg/ml ampicillin. Cultures were grown until the OD₅₉₅ of the cells was between 0.4 and 0.8. Bacterial expression was induced for 3 hours with 1 mM IPTG. Bacterial expression produced an induced band of approximately 15 kDa for muKS1 a and huKS1 a.

MuKS1a and huKS1a were expressed in insoluble inclusion bodies. In order to purify the polypeptides, bacterial cell pellets were re-suspended in lysis buffer (20 mM Tris-HCl pH 8.0, 10 mM βMercaptoethanol, 1 mM PMSF). To the lysed cells, 1% NP-40 was added and the mix incubated on ice for 10 minutes. Lysates were further disrupted by sonication on ice at 95 W for 4×15 seconds and then centrifuged for 10 minutes at 18,000 rpm to pellet the inclusion bodies.

The pellet containing the inclusion bodies was re-suspended in lysis buffer containing 0.5% w/v CHAPS and sonicated for 5-10 seconds. This mix was stored on ice for 1 hour, centrifuged at 14000 rpm for 15 minutes at 4° C. and the supernatant discarded. The pellet was once more re-suspended in lysis buffer containing 0.5% w/v CHAPS, sonicated, centrifuged, and the supernatant removed as before. The pellet was re-suspended in solubilizing buffer (6 M guanidine HCl, 0.5 M NaCl, 20 mM Tris-HCl pH 8.0), sonicated at 95W for 4×15 seconds and centrifuged for 10 minutes at 18000 rpm and 4° C. to remove debris. The supernatant was stored at 4° C. MuKS1 a and huKS1 a were purified by virtue of the N-terminal 6× histidine tag contained within the bacterial leader sequence, using a Nickel-Chelating sepharose column (Amersham Pharmacia, Uppsala, Sweden) and following the manufacturer's protocol. Proteins were purified twice over the column to reduce endotoxin contamination. In order to re-fold the proteins once purified, the protein solution was dialysed in a 4 M-2 M urea gradient in 20 mM tris-HCl pH 7.5+10% glycerol overnight at 4° C. The protein was then further dialysed 2× against 2 liters of 20 mM Tris-HCl pH 7.5+10% (w/v) glycerol. Preparations obtained were greater than 95% pure as determined by SDS-PAGE. Endotoxin contamination of purified proteins were determined using a limulus amebocyte lysate assay kit (BIO Whittaker, Walkersville, Md.). Endotoxin levels were <0.1 ng/μg of protein. Internal amino acid sequencing was performed on tryptic peptides of KS1.

An Fc fusion protein was produced by expression in HEK 293 T cells. 35%g of KLF-1p1GFc DNA to transfect 6×10⁶ cells per flask, 200 mls of Fc containing supernatant was produced. The Fc fusion protein was isolated by chromatography using an Affiprep protein A resin (0.3 ml column, Biorad). After loading, the column was washed with 15 mls of PBS, followed by a 5 ml wash of 50 mM Na citrate pH 5.0. The protein was then eluted with 6 column volumes of 50 mM Na citrate pH 2.5, collecting 0.3 ml fractions in tubes containing 601 μl of 2M Tris-HCl pH 8.0. Fractions were analyzed by SDS-PAGE.

Peptide Sequencing of muKS1 and huKS1

Bacterially expressed muKS1 and huKS1 were separated on polyacrylamide gels and induced bands of 15 kDa were identified. The predicted size of muKS1 is 9.4 kDa. To obtain the amino acid sequence of the 15 kDa bands, 20 μg recombinant muKS1 and huSK1 was resolved by SDS-PAGE and electroblotted onto Immobilon PVDF membrane (Millipore, Bedford, Mass.). Internal amino acid sequencing was performed on tryptic peptides of muKS1 and huKS1 by the Protein Sequencing Unit at the University of Auckland, New Zealand.

The determined amino acid sequences for muKS1 and huKS1 are given in SEQ ID NOS: 397 and 398, respectively. These amino acid sequences confirmed that the determined sequences are identical to those established on the basis of the cDNA sequences. The size discrepancy has previously been reported for other chemokines (Richmond A, Balentien E, Thomas H G, Flaggs G, Barton D E, Spiess J, Bordoni R, Francke U, Derynck R, “Molecular characterization and chromosomal mapping of melanoma growth stimulatory activity, a growth factor structurally related to beta-thromboglobulin,” EMBO J. 7:2025-2033, 1988; Liao F, Rabin R L, Yannelli J R, Koniaris L G, Vanguri P, Farber J M, “Human Nig chemokine: biochemical and functional characterization,” J. Exp. Med. 182:1301-1314, 1995). The isoelectric focusing point of these proteins was predicted to be 10.26 using DNASIS (HITACHI Software Engineering, San Francisco, Calif.). Recombinant Fc tagged KS1 expresssed and purified using protein A affinity column chromatography revealed a homogenous protein with a molecular mass of 42 kDa.

Oxidative Burst Assay

Oxidative burst assays were used to determine responding cell types. 1×10⁷ PBMC cells were resuspended in 5 ml HBSS, 20 mM HEPES, 0.5% BSA and incubated for 30 minutes at 37° C. with 5 μl 5 mM dichloro-dihydrofluorescein diacetate (H₂DCFDA, Molecular Probes, Eugene, Oreg.). 2×10⁵H₂DCFDA-labeled cells were loaded in each well of a flat-bottomed 96 well plate. 10 μl of each agonist was added simultaneously into the well of the flat-bottomed plate to give final concentrations of 100 ng/ml (fMLP was used at 10 μM). The plate was then read on a Victor² 1420 multilabel counter (Wallac, Turku, Finland) with a 485 nm excitation wavelength and 535 nm emission wavelength. Relative fluorescence was measured at 5 minute intervals over 60 minutes.

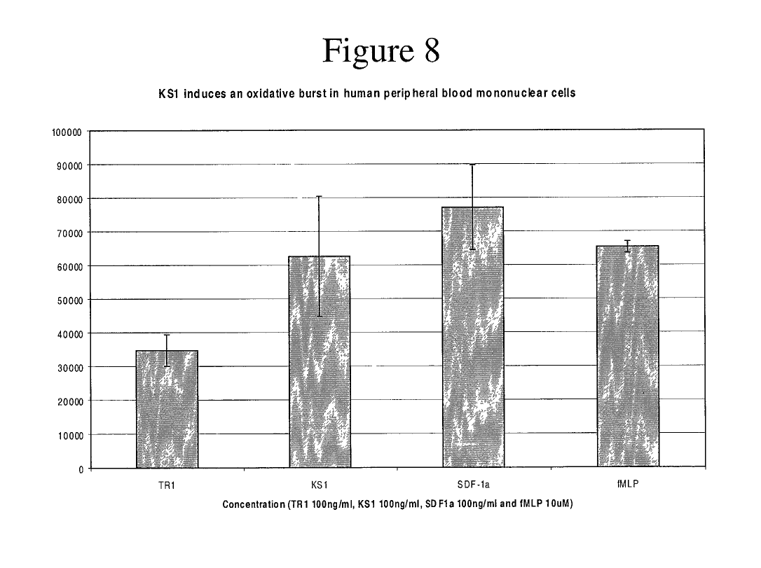

A pronounced respiratory burst was identified in PBMC with a 2.5 fold difference between control treated cells (TR1) and cells treated with 100 ng/ml muKS1 (FIG. 8). Human stromal derived factor-1α(SDF1a) (100 ng/ml) and 10 μM formyl-Met-Leu-Phe (fMLP) were used as positive controls.

Chemotaxis Assay

Cell migration in response to muKS1 was tested using a 48 well Boyden's chamber (Neuro Probe Inc., Cabin John, Maryland) as described in the manufacturer's protocol. In brief, agonists were diluted in HBSS, 20 mM HEPES, 0.5% BSA and added to the bottom wells of the chemotactic chamber. THP-1 cells were re-suspended in the same buffer at 3×10⁵ cells per 50 μl. Top and bottom wells were separated by a PVP-free polycarbonate filter with a 5 μm pore size for monocytes or 3 μm pore size for lymphocytes. Cells were added to the top well and the chamber incubated for 2 hours for monocytes and 4 hours for lymphocytes in a 5% CO₂ humidified incubator at 37° C. After incubation, the filter was fixed and cells scraped from the upper surface. The filter was then stained with Diff-Quick (Dade International Inc., Miami, Fla.) and the number of migrating cells counted in five randomly selected high power fields. The results are expressed as a migration index (the number of test migrated cells divided by the number of control migrated cells).

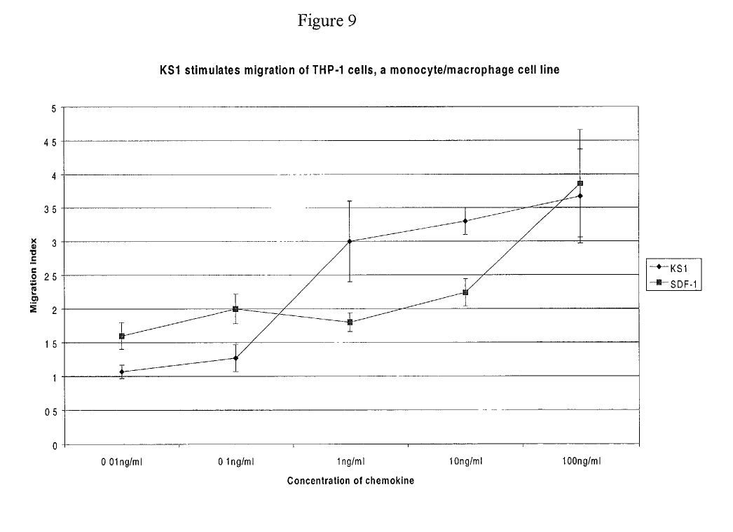

Using this assay, muKS1 was tested against T cells and THP-1 cells. MuKS1 induced a titrateable chemotactic effect on THP-1 cells from 0.01 ng/ml to 100 ng/ml (FIG. 9). Human SDF1α was used as a positive control and gave an equivalent migration. MuKS1 was also tested against IL-2 activated T cells. However, no migration was evidence for muKS1 even at high concentrations, whereas SDF-1α provided an obvious titrateable chemotactic stimulus. Therefore, muKS1 appears to be chemotactic for THP-1 cells but not for IL-2 activated T cells at the concentrations tested.

Flow Cytometric Binding Studies

Binding of KLF-1 to THP-1 and Jurkat cells was tested in the following manner. THP-1 or Jurkat cells (5×10⁶) were resuspended in 3 mls of wash buffer (2% FBS and 0.2% sodium azide in PBS) and pelleted at 4° C., 200× g for 5 minutes. Cells were then blocked with 0.5% mouse and goat sera for 30 minutes on ice. Cells were washed, pelleted, resuspended in 50 μl of KLF-1Fc at 10 μg/ml and incubated for 30 minutes on ice. After incubation, the cells were prepared as before and resuspended in 50 μl of goat anti-human IgG biotin (Southern Biotechnology Associates, AL) at 10 μg/ml and incuated for 30 minutes on ice. Finally, cells were washed, pelleted and resuspended in 50 μl of streptavidin-RPE (Southern Biotechnology Associates, AL) at 10 μg/ml and incuabated for a further 30 minutes on ice in the dark. Cells were washed and resuspended in 250 μl of wash buffer and stained with 1 μl of 10 μg/ml propidium iodide (Sigma) to exclude any dead cells. Purified Fc fragment (10 μg/ml) was used as a negative control in place of KLF-1Fc to determine non-specific binding. Ten thousand gated events were analyzed on log scale using PE filter arrangement with peak transmittance at 575 nm and bandwidth of 10 nm on an Elite cell sorter (Coulter Cytometry).

The respiratory burst and migration assays indicated that KS1 is active on monocytes and not T cells; therefore, the KS 1 Fc fusion protein was tested in a binding study with THP-1 and Jurkat T cells. KS1 Fc showed a marked positive shift on THP-1 cells compared with the Fc fragment alone. In contrast, KS1 demonstrated no positive binding with Jurkat cells in an identical experiment.

Full Length Sequence of muKS1 Clone

The nucleotide sequence of muKS1 was extended by determining the base sequence of additional ESTs. Combination of all the ESTs identified the full-length muKS1 (SEQ ID NO: 370) and the corresponding translated polypeptide sequence in SEQ ID NO: 394.

Analysis of Human RNA Transcripts by Northern Blotting

Northern blot analysis to determine the size and distribution of mRNA for the human homologue of muKS1 was performed by probing human tissue blots (Clontech, Palo Alto, Calif.) with a radioactively labeled probe consisting of nucleotides 1 to 288 of huKS1 (SEQ ID NO: 270). Prehybridization, hybridization, washing, and probe labeling were performed as described in Sambrook, et al., Ibid. mRNA for huKS1 was 1.6 kb in size and was observed to be most abundance in kidney, liver, colon, small intestine, and spleen. Expression could also be detected in pancreas, skeletal muscle, placenta, brain, heart, prostate, and thymus. No detectable signal was found in lung, ovary, and testis.

Analysis of Human RNA Transcripts in Tumor Tissue by Northern Blotting

Northern blot analysis to determine distribution of huKS1 in cancer tissue was performed as described previously by probing tumor panel blots (Invitrogen, Carlsbad, Calif.). These blots make a direct comparison between normal and tumor tissue. mRNA was observed in normal uterine and cervical tissue but not in the respective tumor tissue. In contrast, expression was up-regulated in breast tumor and down-regulated in normal breast tissue. No detectable signal was found in either ovary or ovarian tumors.

Infection of Bacterially Recombinant muKS1 into C3H/HeJ Mice

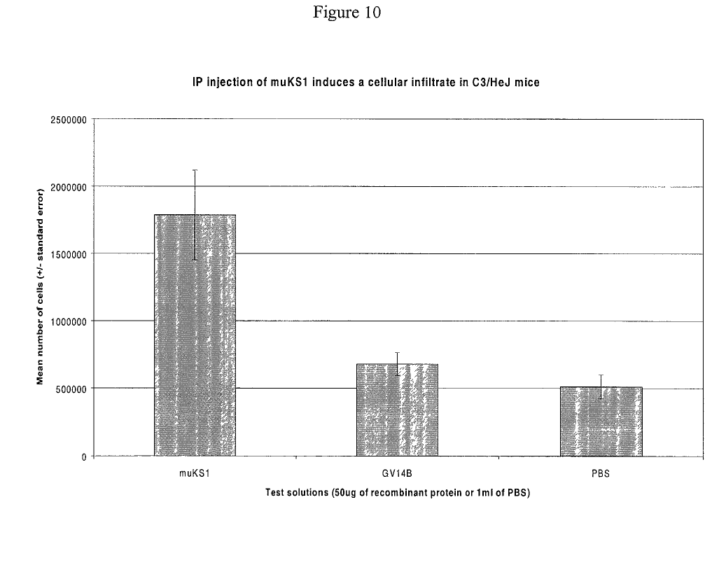

Eighteen C3H/HeJ mice were divided into 3 groups and injected intraperitoneally with muKS1, GV14B, or phosphate buffered saline (PBS). GV14B is a bacterially expressed recombinant protein used as a negative control. Group 1 mice were injected with 50 μg of muKS1 in 1 ml of PBS; Group 2 mice were injected with 50 μg of GV14B in 1 ml of PBS; and Group 3 mice with 1 ml of PBS. After 18 hours, the cells in the peritoneal cavity of the mice were isolated by intraperitoneal lavage with 2×4 ml washes with harvest solution (0.02% EDTA in PBS). Viable cells were counted from individual mice from each group. Mice injected with 50 μg of muKS1 had on average a 3-fold increase in cell numbers (FIG. 10).

20 μg of bacterial recombinant muKS1 was injected subcutaneously into the left hind foot of three C3H/HeJ mice. The same volume of PBS was injected into the same site on the right-hand side of the same animal. After 18 hours, mice were examined for inflammation. All mice showed a red swelling in the foot pad injected with bacterially recombinant KS1. From histology, sites injected with muKS1 had an inflammatory response of a mixed phenotype with mononuclear and polymorphonuclear cells present.

Injection of Bacterially Expressed muKS1a into Nude Mice

To determine whether T cells are required for the inflammatory response, the experiment was repeated using nude mice. Two nude mice were anaesthetised intraperitoneally with 75 l of 1/10 dilution of Hypnorm (Janssen Pharmaceuticals, Buckinghamshire, England) in phosphate buffered saline. 20 ug of bacterially expressed muKS1a (SEQ ID NO: 345) was injected subcutaneously in the left hind foot, ear and left-hand side of the back. The same volume of phosphate buffered saline was injected in the same sites but on the right-hand side of the same animal. Mice were left for 18 hours and then examined for inflammation. Both mice showed a red swelling in the ear and foot sites injected with the bacterially expressed protein. No obvious inflammation could be identified in either back site. Mice were culled and biopsies taken from the ear, back and foot sites and fixed in 3.7% formol saline. Biopsies were embedded, sectioned and stained with Haemotoxylin and eosin. Sites injected with muKS1 a had a marked increase in polymorphonuclear granulocytes, whereas sites injected with phosphate buffered saline had a low background infiltrate of polymorphonuclear granulocytes.

Discussion

Chemokines are a large superfamily of highly basic secreted proteins with a broad number of functions (Baggiolini, et al., Annu. Rev. Immunol., 15:675-705, 1997; Ward, et al., Immunity, 9:1-11, 1998; Horuk, Nature, 393:524-525, 1998). The polypeptide sequences of muKS1 and huKS1 have similarity to CXC chemokines, suggesting that this protein will act like other CXC chemokines. The in vivo data from nude mice supports this hypothesis. This chemokine-like protein may therefore be expected to stimulate leukocyte, epithelial, stromal, and neuronal cell migration; promote angiogenesis and vascular development; promote neuronal patterning, hemopoietic stem cell mobilization, keratinocyte and epithelial stem cell patterning and development, activation and proliferation of leukocytes; and promotion of migration in wound healing events. It has recently been shown that receptors to chemokines act as co-receptors for HIV-1 infection of CD4+ cells (Cairns, et al., Nature Medicine, 4:563-568, 1998) and that high circulating levels of chemokines can render a degree of immunity to those exposed to the HIV virus (Zagury, et al., Proc. Natl. Acad. Sci. USA 95:3857-3861, 1998). This novel gene and its encoded protein may thus be usefully employed as regulators of epithelial, lymphoid, myeloid, stromal, and neuronal cells migration and cancers; as agents for the treatment of cancers, neuro-degenerative diseases, inflammatory autoimmune diseases such as psoriasis, asthma and Crohn's disease for use in wound healing; and as agents for the prevention of HIV-1 binding and infection of leukocytes.

We have also shown that muKS1 promotes a quantifiable increase in cell numbers in the peritoneal cavity of C3H/HeJ mice injected with muKS1. Furthermore, we have shown that muKS1 induces an oxidative burst in human peripheral blood mononuclear cells and migration in the human monocyte leukemia cell line, THP-1, suggesting that monocyte/macrophages are one of the responsive cell types for KS1. In addition to this, we demonstrated that huKS1 was expressed at high levels in a number of non-lymphoid tissues, such as the colon and small intestine, and in breast tumors. It was also expressed in normal uterine and cervical tissue, but was completely down-regulated in their respective tumors. It has recently been shown that non-ELR chemokines have demonstrated angiostatic properties. IP-10 and Mig, two non-ELR chemokines, have previously been shown to be up-regulated during regression of tumors (Tannenbaum C S, Tubbs R, Armstrong D, Finke J H, Bukowski R M, Hamilton TA, “The CXC Chemokines IP-10 and Mig are necessary for IL-12-mediated regression of the mouse RENCA tumor,” J. Immunol. 161: 927-932, 1998), with levels of expression inversely correlating with tumor size (Kanegane C, Sgadari C, Kanegane H, Teruya-Feldstine J, Yao O, Gupta G, Farber J M, Liao F, Liu L, Tosato G, “Contribution of the CXC Chemokines IP-10 and Mig to the antitumor effects of IL-12,” J. Leuko. Biol. 64: 384-392, 1998). Furthermore, neutralizing antibodies to IP-10 and Mig would reduce the anti-tumor effect, indicating the contribution these molecules make to the anti-tumor effects. Therefore, it is expected that in the case of cervical and uterine tumors, KS1 would have similar properties.

The data demonstrates that KS1 is involved in cell migration showing that one of the responsive cell types is monocyte/macrophage. The human expression data in conjunction with the in vitro and in vivo biology demonstrates that this molecule may be a useful regulator in cell migration, and as an agent for the treatment of inflammatory diseases, such as Crohn's disease, ulcerative colitis, and rheumatoid arthritis; and cancers, such as cervical adenocarcinoma, uterine leiomyoma, and breast invasive ductal carcinoma.

### EXAMPLE 6

### Characterization of KS2

KS2 contains a transmembrane domain and may function as either a membrane-bound ligand or a receptor. Northern analysis indicated that the mRNA for KS2 was expressed in the mouse keratinocyte cell line, Pam212, consistent with the cDNA being identified in mouse keratinocytes.

Mammalian Expression

To express KS2, the extracellular domain was fused to the amino terminus of the constant domain of immunoglobulinG (Fc) that had a C-terminal 6× Histidine tag. This was performed by cloning polynucleotides 20-664 of KS2 (SEQ ID NO: 273), encoding amino acids 1-215 of polypeptide KS2 (SEQ ID NO: 347), into the mammalian expression vector pcDNA3 (Invitrogen, NV Leek, Netherlands), to the amino terminus of the constant domain of immunoglobulinG (Fc) that had a C-terminal 6× Histidine tag. This construct was transformed into competent XL1-Blue E.coli as described in Sambrook et al., Ibid. The Fc fusion construct of KS2a was expressed by transfecting Cos-1 cells in 5× T175 flasks with 180 μg of KS1a using DEAE-dextran. The supernatant was harvested after seven days and passed over a Ni-NTA column. Bound KS2a was eluted from the column and dialysed against PBS.

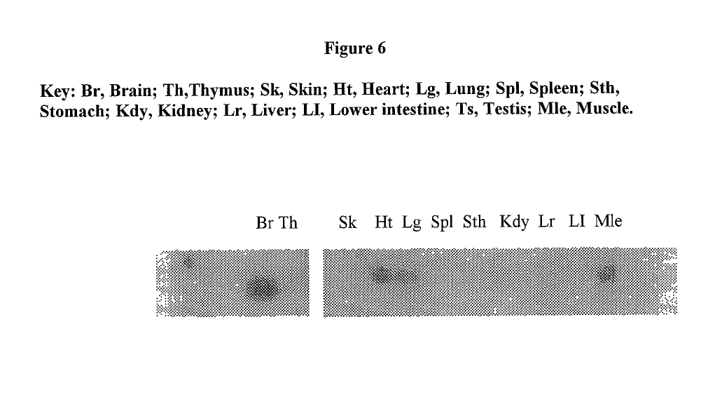

The ability of the Fc fusion polypeptide of KS2a to inhibit the IL-2 induced growth of concanavalin A stimulated murine splenocytes was determined as follows. A single cell suspension was prepared from the spleens of BALB/c mice and washed into DMEM (GIBCO-BRL) supplemented with 2 mM L-glutamine, 1 mM sodium pyruvate, 0.77 mM L-asparagine, 0.2 mM L-arganine, 160 mM penicillin G, 70 mM dihydrostreptomycin sulfate, 5×10⁻² mM beta mercaptoethanol and 5% FCS (cDMEM). Splenocytes (4×10⁶/nil) were stimulated with 2 μg/ml concanavalin A for 24 hrs at 37° C. in 10% CO₂. The cells were harvested from the culture, washed 3 times in cDMEM and resuspended in cDMEM supplemented with 10 ng/ml rhuIL-2 at 1×10⁵ cells/ml. The assay was performed in 96 well round bottomed plates in 0.2 ml cDMEM. The Fc fusion polypeptide of KS2a, PBS, LPS and BSA were titrated into the plates and 1×10⁴ activated T cells (0.1 ml) were added to each well. The plates were incubated for 2 days in an atmosphere containing 10% CO₂ at 37° C. The degree of proliferation was determined by pulsing the cells with 0.25 uCi/ml tritiated thymidine for the final 4 hrs of culture after which the cells were harvested onto glass fiber filtermats and the degree of thymidine incorporation determined by standard liquid scintillation techniques. As shown in FIG. 6, the Fc fusion polypeptide of KS2a was found to inhibit the IL-2 induced growth of concanavalin A stimulated murine splenocytes, whereas the negative controls PBS, BSA and LPS did not.

This data demonstrates that KS2 is expressed in skin keratinocytes and inhibits the growth of cytokine induced splenocytes. This indicates a role for KS2 in the regulation of skin inflammation and malignancy.

### EXAMPLE 7

### Characterization of KS3

KS3 encodes a polypeptide of 40 amino acids (SEQ ID NO: 129). KS3 contains a signal sequence of 23 amino acids that would result in a mature polypeptide of 17 amino acids (SEQ ID NO: 348; referred to as KS3a).

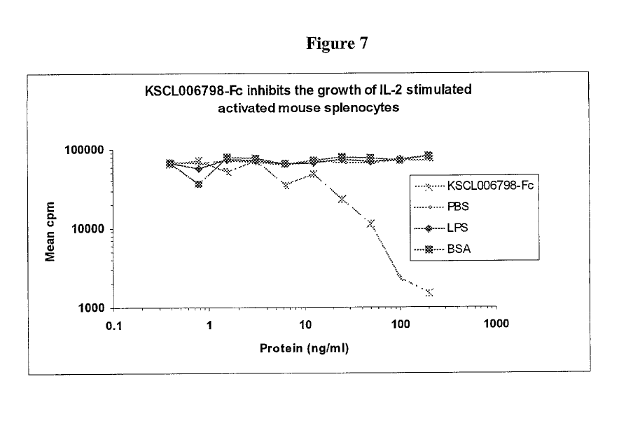

KS3a was prepared synthetically (Chiron Technologies, Victoria, Australia) and observed to enhance transferrin-induced growth of the rat intestinal epithelial cells IEC-18 cells. The assay was performed in 96 well flat-bottomed plates in 0.1 ml DMEM (GIBCO-BRL Life Technologies) supplemented with 0.2% FCS. KS3a (SEQ ID NO: 348), apo-Transferrin, media and PBS-BSA were titrated either alone, with 750 ng/ml Apo-transferrin or with 750 ng/ml BSA, into the plates and 1×10³ IEC-18 cells were added to each well. The plates were incubated for 5 days at 37° C. in an atmosphere containing 10% CO₂. The degree of cell growth was determined by MTT dye reduction as described previously (J. Imm. Meth. 93:157-165, 1986). As shown in FIG. 7, KS3a plus Apo-transferrin was found to enhance transferrin-induced growth of IEC-18 cells, whereas KS3a alone or PBS-BSA did not, indicating that Ks3a and Apo-transferrin act synergistically to induce the growth of IEC-18 cells.

This data indicates that KS3 is epithelial derived and stimulates the growth of epithelial cells of the intestine. This suggests a role for KS3 in wound healing, protection from radiation- or drug-induced intestinal disease, and integrity of the epithelium of the intestine.

SEQ ID NOS: 1-725 are set out in the attached Sequence Listing. The codes for polynucleotide and polypeptide sequences used in the attached Sequence Listing confirm to WIPO Standard ST.25 (1988), Appendix 2.

All references cited herein, including patent references and non-patent references, are hereby incorporated by reference in their entireties.

Although the present invention has been described in terms of specific embodiments, changes and modifications can be carried out without departing from the scope of the invention which is intended to be limited only by the scope of the appended claims.

We claim:

1. An isolated polynucleotide comprising a nucleotide sequence selected from the group consisting of (a) sequences recited in SEQ ID NOS: 1-119, 198-276, 349-372, 399-405, 410-412, 416, 418-455, 464, 466-487, 510, 511 and 514-623; (b) complements of the sequences recited in SEQ ID NOS: 1-119, 198-276, 349-372, 399-405, 410-412, 416, 418-455, 464, 466-487, 510, 511 and 514-623; (c) reverse complements of the sequences recited in SEQ ID NOS: 1-119, 198-276, 349-372, 399-405, 410-412, 416, 418-455, 464, 466-487, 510, 511 and 514-623; (d) reverse sequences of the sequences recited in SEQ ID NOS: 1-119, 198-276, 349-372, 399-405, 410-412, 416, 418-455, 464, 466-487, 510, 511 and 514-623; (e) sequences having at least a 99% probability of being the same as a sequence selected from any of the sequences in (a)-(d), above, as measured by the computer algorithm BLASTP using the running parameters described above; (f) nucleotide sequences having at least 75% identity to any of the sequences in (a)-(d), above, as measured by the computer algorithm BLASTP using the running parameters and identity test defined above; (g) nucleotide sequences having at least 90% identity to any of the sequences in (a)-(d), above, as measured by the computer algorithm BLASTP using the running parameters and identity test defined above; (h) nucleotide sequences having at least 95% identity to any of the sequences in (a)-(d), above, as measured by the computer algorithm BLASTP using the running parameters and identity test defined above; and (g) open reading frames of SEQ ID NOS: 1-119, 198-276, 349-372, 399-405, 410-412, 416, 418-455, 464, 466-487, 510, 511 and 514-623.

2. An expression vector comprising an isolated polynucleotide of claim 1.

3. A host cell transformed with an expression vector of claim 2.

4. An isolated polypeptide comprising an amino acid sequence selected from the group consisting of (a) sequences provided in SEQ ID NOS: 120-197, 275-348, 373-398, 406-409, 413-415, 417, 456-463, 465, 488-509, 512, 513 and 624-725; (b) sequences having at least a 99% probability of being the same as a sequence of SEQ ID NOS: 120-197, 275-348, 373-398, 406-409, 413-415, 417, 456-463, 465, 488-509, 512, 513 and 624-725, as measured by the computer algorithm BLASTP using the running parameters described above; (c) sequences having at least 75% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP using the running parameters and identity test defined above; (d) sequences having at least 90% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP using the running parameters and identity test defined above; (e) sequences having at least 95% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP using the running parameters and identity test defined above; and (f) sequences encoded by a sequence provided in SEQ ID NOS: 1-119, 198-276, 349-372, 399-405, 410-412, 416, 418-455, 464, 466-487, 510, 511 and 514-623.

5. An isolated polypeptide comprising amino acids 54-104 of SEQ ID NO: 196.

6. An isolated polynucleotide encoding a polypeptide of claim 4.

7. An expression vector comprising an isolated polynucleotide of claim 6.

8. A host cell transformed with an expression vector of claim 7.

9. An isolated polypeptide comprising at least a functional portion of a polypeptide having an amino acid sequence selected from the group consisting of (a) sequences provided in SEQ ID NOS: 120-197, 275-348, 373-398, 406-409, 413-415, 417, 456-463, 465, 488-509, 512, 513 and 624-725; (b) sequences having at least a 99% probability of being the same as a sequence of SEQ ID NOS: 120-197, 275-348, 373-398, 406-409, 413-415, 417, 456-463, 465, 488-509, 512, 513 and 624-725, as measured by the computer algorithm BLASTP using the running parameters described above; (c) sequences having at least 75% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP, using the running parameters and identity test defined above; (d) sequences having at least 90% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP, using the running parameters and identity test defined above; (e) sequences having at least 95% identity to a sequence provided in SEQ ID NOS: 120-197, 275-348, 373-398, 406-409, 413-415, 417, 456-463, 465, 488-509, 512, 513 and 624-725, as measured by the computer algorithm BLASTP, using the running parameters and identity test defined above; and (f) sequences encoded by a sequence provided in SEQ ID NOS: 1-119, 198-276, 349-372, 399-405, 410-412, 416, 418-455, 464, 466-487, 510, 511 and 514-623.

10. A method for stimulating keratinocyte growth and motility in a patient, comprising administering to the patient a composition comprising a polypeptide of any one of claims 4 and 5.

11. The method of claim 10, wherein the polypeptide comprises an amino acid sequence selected from the group consisting of (a) SEQ ID NOS: 187, 196, 342, 343, 395, 397 and 398; (b) sequences having at least about 75% identity to a sequence of SEQ ID NOS: 187, 196, 342, 343, 395, 397 and 398 as measured by the computer algorithm BLASTP using the running parameters and identity test defined above; (c) sequences having at least about 90% identity to a sequence of SEQ ID NOS: 187, 196, 342, 343, 395, 397 and 398 as measured by the computer algorithm BLASTP using the running parameters and identity test defined above; and (d) sequences having at least about 95% identity to a sequence of SEQ ID NOS: 187, 196, 342, 343, 395, 397 and 398 as measured by the computer algorithm BLASTP using the running parameters and identity test defined above.

12. A method for inhibiting the growth of cancer cells in a patient, comprising administering to the patient a composition comprising a polypeptide of any one of claims 4 and 5.

13. The method of claim 12, wherein the polypeptide comprises an amino acid sequence selected from the group consisting of: (a) SEQ ID NOS: 187, 196, 342, 343, 397 and 398; (b) sequences having at least 75% identity to a sequence of SEQ ID NOS: 187, 196, 342, 343, 397 and 398, as measured by the computer algorithm BLASTP using the running parameters and identity test defined above; (c) sequences having at least 90% identity to a sequence of SEQ ID NOS: 187, 196, 342, 343, 397 and 398, as measured by the computer algorithm BLASTP using the running parameters and identity test defined above; and (d) sequences having at least 95% identity to a sequence of SEQ ID NOS: 187, 196, 342, 343, 397 and 398, as measured by the computer algorithm BLASTP using the running parameters and identity test defined above.

14. A method for modulating angiogenesis in a patient, comprising administering to the patient a composition comprising a polypeptide of any one of claims 4 and 5.

15. The method of claim 14, wherein the polypeptide comprises an amino acid sequence selected from the group consisting of (a) SEQ ID NOS: 187, 196, 342, 343, 397 and 398; (b) sequences having at least 75% identity to a sequence of SEQ ID NOS: 187, 196, 342, 343, 397 and 398 as measured by the computer algorithm BLASTP using the running parameters and identity test defined above; (c) sequences having at least 90% identity to a sequence of SEQ ID NOS: 187, 196, 342, 343, 397 and 398 as measured by the computer algorithm BLASTP using the running parameters and identity test defined above; and (d) sequences having at least 95% identity to a sequence of SEQ ID NOS: 187, 196, 342, 343, 397 and 398 as measured by the computer algorithm BLASTP using the running parameters and identity test defined above.

16. A method for inhibiting angiogenesis and vascularization of tumors in a patient, comprising administering to a patient a composition comprising a polypeptide of any one of claims 4 and 5.

17. The method of claim 16, wherein the polypeptide comprises an amino acid sequence selected from the group consisting of (a) SEQ ID NOS: 187, 196, 342, 343, 397 and 398; (b) sequences having at least 75% identity to a sequence of SEQ ID NOS: 187, 196, 340, 342-346, 397 and 398, as measured by the computer algorithm BLASTP using the running parameters and identity test defined above; (c) sequences having at least 90% identity to a sequence of SEQ ID NOS: 187, 196, 340, 342-346, 397 and 398, as measured by the computer algorithm BLASTP using the running parameters and identity test defined above; and (d) sequences having at least 95% identity to a sequence of SEQ ID NOS: 187, 196, 340, 342-346, 397 and 398, as measured by the computer algorithm BLASTP using the running parameters and identity test defined above.

18. A method for modulating skin inflammation in a patient, comprising administering to the patient a composition comprising a polypeptide of any one of claims 4 and 5.

19. The method of claim 18, wherein the polypeptide comprises an amino acid sequence selected from the group consisting of: (a) SEQ ID NOS: 338 and 347; and (b) sequences having at least 75% identity to a sequence of SEQ ID NOS: 338 and 347 as measured by the computer algorithm BLASTP using the running parameters and identity test defined above; (c) sequences having at least 90% identity to a sequence of SEQ ID NOS: 338 and 347 as measured by the computer algorithm BLASTP using the running parameters and identity test defined above; and (d) sequences having at least 95% identity to a sequence of SEQ ID NOS: 338 and 347 as measured by the computer algorithm BLASTP using the running parameters and identity test defined above.

20. A method for stimulating the growth of epithelial cells in a patient, comprising administering to the patient a composition comprising a polypeptide of claim 4.

21. The method of claim 20, wherein the polypeptide comprises an amino acid sequence selected from the group consisting of: (a) SEQ ID NOS: 129 and 348; (b) sequences having at least 75% identity to a sequence of SEQ ID NOS: 129 and 348 as measured by the computer algorithm BLASTP using the running parameters and identity test defined above; (c) sequences having at least 90% identity to a sequence of SEQ ID NOS: 129 and 348 as measured by the computer algorithm BLASTP using the running parameters and identity test defined above; and (d) sequences having at least 95% identity to a sequence of SEQ ID NOS: 129 and 348 as measured by the computer algorithm BLASTP using the running parameters and identity test defined above.

22. A method for inhibiting the binding of HIV-1 to leukocytes in a patient, comprising administering to the patient a composition comprising a polypeptide of claim 4.

23. The method of claim 22, wherein the polypeptide comprises an amino acid sequence selected from the group consisting of: (a) SEQ ID NOS: 340, 344, 345, 346 and 465; (b) sequences having at least 75% identity to a sequence of SEQ ID NOS: 340, 344, 345, 346 and 465 as measured by the computer algorithm BLASTP using the running parameters and identity test defined above; (c) sequences having at least 90% identity to a sequence of SEQ ID NOS: 340, 344, 345, 346 and 465 as measured by the computer algorithm BLASTP using the running parameters and identity test defined above; and (d) sequences having at least 95% identity to a sequence of SEQ ID NOS: 340, 344, 345, 346 and 465 as measured by the computer algorithm BLASTP using the running parameters and identity test defined above.

24. A method for treating an inflammatory disease in a patient, comprising administering to the patient a composition comprising a polypeptide of claim 4.

25. The method of claim 24, wherein the polypeptide comprises an amino acid sequence selected from the group consisting of (a) SEQ ID NOS: 340, 344, 345, 346 and 465; (b) sequences having at least 75% identity to a sequence of SEQ ID NOS: 340, 344, 345, 346 and 465 as measured by the computer algorithm BLASTP using the running parameters and identity test defined above; (c) sequences having at least 90% identity to a sequence of SEQ ID NOS: 340, 344, 345, 346 and 465 as measured by the computer algorithm BLASTP using the running parameters and identity test defined above; and (d) sequences having at least 95% identity to a sequence of SEQ ID NOS: 340, 344, 345, 346 and 465 as measured by the computer algorithm BLASTP using the running parameters and identity test defined above.

26. A method for treating cancer in a patient, comprising administering to the patient a composition comprising a polypeptide of claim 4.

27. The method of claim 26, wherein the polypeptide comprises an amino acid sequence selected from the group consisting of (a) SEQ ID NOS: 340, 344, 345, 346 and 465; (b) sequences having at least 75% identity to a sequence of SEQ ID NOS: 340, 344, 345, 346 and 465 as measured by the computer algorithm BLASTP using the running parameters and identity test defined above; (c) sequences having at least 90% identity to a sequence of SEQ ID NOS: 340, 344, 345, 346 and 465 as measured by the computer algorithm BLASTP using the running parameters and identity test defined above; and (d) sequences having at least 95% identity to a sequence of SEQ ID NOS: 340, 344, 345, 346 and 465 as measured by the computer algorithm BLASTP using the running parameters and identity test defined above.

28. A method for treating a neurological disease in a patient, comprising administering to the patient a composition comprising a polypeptide of any one of claims 4 and 5.

29. The method of claim 28, wherein the polypeptide comprises an amino acid sequence selected from the group consisting of (a) SEQ ID NOS: 187, 196, 340, 342-346, 397 and 398; (b) sequences having at least 75% identity to a sequence of SEQ ID NOS: 187, 196, 340, 342-346, 397 and 398, as measured by the computer algorithm BLASTP using the running parameters and identity test defined above; (c) sequences having at least 90% identity to a sequence of SEQ ID NOS: 187, 196, 340, 342-346, 397 and 398, as measured by the computer algorithm BLASTP using the running parameters and identity test defined above; and (d) sequences having at least 95% identity to a sequence of SEQ ID NOS: 187, 196, 340, 342-346, 397 and 398, as measured by the computer algorithm BLASTP using the running parameters and identity test defined above.
